(19) United States
(12) Patent Application Publication
(10) Pub. No.: US 2019/0328820 A1
(43) Pub. Date: Oct. 31, 2019

(54) COMPOSITION COMPRISING A PLANT EXTRACT OF THE SPECIE HEDYCHIUM CORONARIUM, FOR USE IN A METHOD FOR TREATMENT OF THE HUMAN BODY BY THERAPY

(71) Applicant: SOCIETE D'EXPLOITATION DE PRODUITS POUR LES INDUSTRIES CHIMIQUES SEPPIC, Paris (FR)

(72) Inventor: Virginie ANCHARTECHAHAR, LONS (FR)

(21) Appl. No.: 16/475,282

(22) PCT Filed: Dec. 8, 2017
(86) PCT No.: PCT/EP2017/081977

§ 371 Date: Jul. 1, 2019

(30) Foreign Application Priority Data
Dec. 30, 2016 (EP) 16207612.9

(51) Int. Cl. A61K 36/906

(52) U.S. Cl. CPC A61K 36/906; A61K 2236/53; A61K 2236/333

(57) ABSTRACT

Disclosed is a composition including a plant extract of the specie Hedychium coronarium, for use in a method for treatment of the human body by therapy; and more particularly for use in protecting and/or activating at least one of the cellular clean-up system.

The present invention relates to the use of a plant extract of the specie Hedychium coronarium in applications for the treatment of skin affected by harmful environmental influences. The invention further relates to a process for preparing said extract.

Hedychium is a genus of flowering plants in the ginger family Zingiberaceae. There are approximately seventy to eighty known species, native to Southeast Asia (Thailand, Malaysia, Indonesia, the Philippines, etc.), southern China, the Himalayas and Madagascar. Some species have become widely naturalized in other lands (South Africa, South America, Central America, the West Indies, and many of the islands of the Pacific, Indian and Atlantic Oceans). The genus name Hedychium is derived from two ancient Greek words, hedys meaning “sweet” and chios meaning “snow”. This refers to the fragrant white flower of the type species Hedychium coronarium. Common names include garland flower, ginger lily, and kahili ginger.

Members of the genus Hedychium are rhizomatous perennials, commonly growing from 120 cm to 180 cm tall. Some species are cultivated for their exotic foliage and fragrant spikes of flowers in shades of white, yellow and orange. Numerous cultivars have been developed for garden use. Selected species comprise for example Hedychium aurantiacum, Hedychium coccineum, Hedychium coronarium, Hedychium densiflorum, Hedychium ellipticum (shaving brush ginger), Hedychium flavescens, Hedychium gardnerianum (ginger lily), Hedychium samuiense, and Hedychium spicatum called kapur kachari in Hindi).

The individual species differ from each other in their individual biological taxonomy and is several cases also with respect to their origin.

The species Hedychium coronarium (also known as “ginger butterfly”) was first described in 1783 by Johann Gerhard Koenig in the book of Andrea Johan Retzius “Observationes Botanicae” t. 3 pages 73-74.

The species Hedychium spicatum (also known as “wild-headed ginger”) was first recorded by James Edward Smith (1811), and then described by Francis Buchanan-Hamilton in 1819 in the work of Abraham REES “the Cyclopaedia; universal dictionary Arts, Sciences and Literature.” t. 17 p. 521-522.

Hedychium has widely been described for use in traditional medicine as described for example in “Edible Medicinal and non medicinal Plants”, Vol. 8 Flowers, pages 853-860” or in “Medicinal plants used by women from Agnalazaha littoral forest (South-eastern Madagascar)”, Journal of Ethno biology and Ethno medicine, 2013 as well as by X. Yan, et al. “Traditional Chinese Medicines, Molecular Structures, Natural Sources, and Applications”, 1999 or by Sharma et al. “Phytochemistry”, 14:578, 1975.

WO 2002/056859 A2 discloses relates to compositions comprising Hedychium extract and the cosmetic use thereof. However, said international application particularly describes the use of the species Hedychium spicatum and in one aspect its activity in regulating the firmness, tone, or texture of skin and in another aspect its use in the treatment of environmental damage in skin based on the underlying mechanisms of inhibiting UV induced metalloproteinase-1 (MMP-1) secretion, preventing smoke-induced loss of thiols to protect the glutathione as a part of the endogenous cellular antioxidant defence system, and inhibiting nitric oxide production as a precursor for the formation of harmful reactive oxygen species (ROS).

BR P10905586-0 A2 describes the cosmetic use of Hedychium coronarium extracts derived from the flowers of the plant for moisturizing, revitalizing, and regenerating the skin.

Further, an anti-aging effect due to high concentration of flavonoids in the flowers is mentioned as well as an anti-inflammatory and anti-peroxidant activity, and a strengthening of micro vessels and capillaries, and the property of combating the formation of oedemas and photo-induced erythemas.

WO 2006/053415 A1 mentions the suitability of several plant extracts in cosmetic applications, including applications making use of the mechanism of inhibition of MMP-1, MMP-2, MMP-3, MMP-9, HLE (human leukocyte elastase). However, therein Hedychium plant extracts are only mentioned very generally in a large list of possible plants and no specific effect has been described to or shown for any Hedychium species.

According to a first embodiment, the invention relates to a composition comprising a plant extract of the specie Hedychium coronarium, for use in a method for treatment of the human body by therapy, such as in particular for the protection against and/or the treatment of environmental damages of the skin, and more particularly for use in protecting and/or activating at least one of the cellular clean-up system in a method for treatment of the human body by therapy.

By environmental damage, It means any harmful environmental influences such as harmful influences from toxins and/or pollutants, including exhaust, industrial pollution, agricultural pollution, and cigarette smoke, as well as harmful radiation, such as UV radiation, e.g., from the sun or non-natural sources including UV lamps and solar simulators, and ozone.

As used herein, “protection against environmental damage” means the prevention and/or reduction of symptoms or of the harmful effects deriving from the harmful environmental influences.

As used herein, “treatment of environmental damage” means the reduction, amelioration, improvement and/or elimination of symptoms or damages deriving from the harmful environmental influences.

As used herein, the cellular clean-up system comprises cellular systems of the cell metabolism (hereinafter also referred to as “cellular metabolic system”) and of the intracellular barrier system, which are essential for providing viable and intact cells and which constitutes a further key element of viable and vital cells and a functioning cellular defence system against harmful environmental influences. Therein, cellular systems of the cellular metabolic system and of the intracellular barrier system comprise in particular the following systems:
The mitochondria which generate most of the cell's supply of adenosine triphosphate (ATP), are used as a source of chemical energy and thus represent the so-called power supply of the cells. In addition to supplying cellular energy, mitochondria are involved in a range of other crucial cell processes, such as signalling, thermo genesis, cellular differentiation, control of the cell division cycle and cell growth, cell death (apoptosis), oxidative metabolism, homeostasis of glucids, lipids, calcium, iron (heme synthesis), and much more.
The lysosomes are organelles of the cytoplasm that permit recycling of cellular materials that have exceeded their lifetime or are otherwise no longer useful. Their main function is the digestion of endogenous or exogenous substrates (so called autophagy or heterophagy), in all eukaryotic cells. Lysosomes break down cellular waste products, fats, carbohydrates, proteins, and other macromolecules into simple compounds, which are then transferred back into the cytoplasm as new cell-building materials. Indeed, its lipid membrane contains many enzymes (about 40 different types of hydrolytic enzymes), protons pumps and transport proteins. Acid pH is regulated to allow optimum activities of acid hydrolases (so a lysosome is like a cell stomach). Autophagy is an important mechanism that allows the cell to mobilize its energy stocks to defend and destroy its damaged organelles and then avoid serious effects. This process permits elimination and replacement of proteins and non-functional organelles, then to ensure homeostasis. It is involved in longevity control and development of pathologies as cancer or diabetes.
The intracellular barrier system which in particular comprises the cellular transmembrane system, plays an important role in the cellular clean-up system of the cells, as the cellular transmembrane system controls and effects the transport of the metabolic energy products from the mitochondria and of the metabolic waste products from the lysosomes and thus plays a key role in an effective and functioning cell metabolism which are essential for providing viable and intact cells. A strong and intact transmembrane system comprises so called “tight junctions”, i.e. junctions established between cells (cell-cell junctions). These junctions are made of several families of transmembrane proteins, such as claudins, occludin and adhesion molecules (ZO-1, ZO-2, ZO-3, . . . ). Tight junctions exist in skin tissue, including epidermis layers. Claudins 1 and 4 are proteins found in upper layers of epidermis (where keratinocytes differentiate). Immunostainings studies have shown the presence of these markers and that these proteins participate in paracellular skin barrier by controlling the flow of molecules in the intercellular space between the cells of an epithelium, as well as by blocking the entry of small molecules and by maintaining the integrity (cohesion reinforced between corneocytes) of the epidermis' upper layers. Furthermore, claudins play a role in the homeostasis of the stratum corneum and the control of the calcium gradient. Damage of the tight junctions leads to damage of the skin barrier due to increasing calcium flux and disturbed gradient, leading to an altered differentiation and finally to a damage of the skin barrier protection.
A further important aspect of the cellular metabolic system relates to the production of interleukin 8 (IL-8), a chemokine produced by macrophages and other cell types such as epithelial cells and endothelial cells. Endothelial cells store IL-8 in their storage vesicles, the Weibel-Palade bodies. IL-8 is initially produced as a precursor peptide of 99 amino acids long which then undergoes cleavage to create several active IL-8 isoforms. IL-8 is secreted and is an important mediator of the immune reaction in the innate immune system response. IL-8, also known as neutrophil chemotactic factor, has two primary functions. It induces chemotaxis in target cells, primarily neutrophils but also other granulocytes, causing them to migrate toward the site of infection. IL-8 also induces phagocytosis once they have arrived. IL-8 is also known to be a potent promoter of angiogenesis. In target cells, IL-8 induces a series of physiological responses required for migration and phagocytosis, such as increases in intracellular Ca²⁺, exocytosis (e.g. histamine release), and the respiratory burst. IL-8 can be secreted by any cells with toll-like receptors that are involved in the innate immune response. Usually, the macrophages see an antigen first, and thus are the first cells to release IL-8 in order to recruit other cells. Both monomer and homodimer forms of IL-8 have been reported to be potent inducers of the chemokine receptors CXCR1 and CXCR2.
A further important aspect of the cellular metabolic system relates to the production of β-endorphins. β-Endorphins are neuromediators, endogenous opioids, derived from Propiomelanocortin (POMC) that are involved in various biological phenomena as skin physiology and homeostasis, neurotrophic activity, pain, immune defence, endocrinal, emotional and stress responses In skin, β-endorphins are secreted by keratinocytes and its receptors are present in main skin cells (keratinocytes, fibroblasts and melanocytes). Therewith, the β-endorphins are said to play an important role in various skin metabolism and skin defence processes, comprising stimulation of keratinocyte migration, involvement in wound healing and cellular differentiation, induction of epidermal and follicular melanogenesis, and control of hair growth. Furthermore, increased β-endorphin modulates the number of dendritic processes of hair follicle melanocytes. It becomes there from apparent, that a functioning cellular metabolic system further requires a functioning β-endorphin production system plays a further important role in the cellular clean-up system of the cells, thus being a further essential aspect for providing viable and vital cells and a functioning cellular defence system against harmful environmental influences.
Besides the aforementioned aspects of the metabolic system a further aspect relates to the melanogenesis of cells, i.e. the melanin production in the cells by melanocytes. Melanin is a pigment found in the skin, eyes, and hair, thus being responsible of the skin colour. Melanocytes are located in the basal layer of epidermis. The melanocytes are dendritic cells that are connected to the keratinocytes in which they transmit specific organelles, melanosomes, containing melanin. Melanogenesis comprises two steps: melanin synthesis and then transfer to keratinocytes. Melanin is synthesized in the melanosomes of melanocytes, organelles that come from the Golgi apparatus and rough endoplasmic reticulum. The synthesis of melanin is made from the amino acid tyrosine, catalyzed by the tyrosinase enzyme. Tyrosine reacts to Dihydroxyphenylalanine (DOPA), which is then oxidized to Dopaquinon. After several further chemical reaction steps, finally melanin is formed. Within the melanocytes, the melanin pigment is trapped in “bags”, the melanosomes that are then sent to the surface. The melanocytes have dendritic extensions allowing them to come into contact with several keratinocytes. The melanosomes are transported from the cell body where they are produced to the end of the dendrites where they accumulate and are transferred to adjacent keratinocytes where melanin is dispersed. Usually, melanogenesis leads to a long-lasting pigmentation, which is in contrast to the pigmentation that originates from oxidation of already-existing melanin. There are both basal and activated levels of melanogenesis. In general, lighter-skinned people have low basal levels of melanogenesis. Exposure to UV-B radiation causes an increased melanogenesis. The purpose of the melanogenesis is to protect the hypodermis, the layer under the skin, from the UV-B light that can damage it (DNA photo damage). The colour of the melanin is dark, allowing it to absorb a majority of the UV-B light and block it from passing through this skin layer. Numerous stimuli are able to alter the melanogenesis or the production of melanin by cultured melanocytes, although the method by which it works is not fully understood. Also harmful environmental influences such as toxic pollutants and UV irradiation may trigger melanogenesis and, in turn, pigmentation. With regard to its protective function, a functioning melanogenesis desirably provides a constant and uniform pigmentation of the skin. Due to harmful environmental influences the melanogenesis of vital cells may be deteriorated and lead to the formation of undesired spot-like pigmentation and also to the formation of melanoma (also known as malignant melanoma), which is the most dangerous type of skin cancer and that develops from the pigment-containing melanocytes. The primary cause of melanoma is ultraviolet light (UV) exposure in those with low levels of skin pigment. It becomes there from apparent, that a functioning cellular metabolic system further requires a vital and functioning melanogenesis system, leading to a constant and uniform protective pigmentation of skin and avoiding undesired spot-like pigmentation and in particular the formation of melanoma. Thus, a functioning melanogenesis plays a further important role in the cellular clean-up system of the cells, thus being a further essential aspect for providing viable and vital cells and a functioning cellular defence system against harmful environmental influences.

From the aforesaid explanations it is in particular apparent that vital and functioning cells require a strong cellular clean-up system for a vital and functioning cellular defence system against harmful environmental influences, wherein such cellular clean-up system is in particular based on a vital and functioning cellular metabolic system with an optimal interaction of the key elements of functioning mitochondria, lysosomes, IL-8 production, β-endorphin production, melanogenesis and a strong and intact cellular transmembrane system, which according to the present invention together form the cellular clean-up system of the cells.

The invention therefore relates to a composition as hereinbefore defined, in which said clean-up system is selected from, the mitochondrial protection and/or activation, the lysosomal protection and/or activation, the inhibition of cellular IL-8 release, the cellular production of β-endorphins, the inhibition of irregular melanin release, and the protection and/or activation of the cellular transmembrane system by claudin restoration.

In the context of the present invention the term “extract” means an agent derived by extraction of similar or different parts of the plant of the specie Hedychium coronarium plant. An extract in accordance with the present invention is a blend of compounds isolated from the extracted part(s) of the plant.

According to a particular embodiment, said plant extract of the specie Hedychium coronarium, is derived from flowers, seeds, fruits, leaves, stem, roots and/or rhizomes of the plant; and is more particularly derived from roots and/or rhizomes of the plant.

The composition of the present invention may further comprise at least one solvent and/or at least one topically acceptable auxiliary substance.

Examples of suitable solvents include lower C₁-C₈-alcohols C₁-C₈-alkyl polyols, C₁-C₈-alkyl ketones, C₁-C₈-alkyl ethers, acetic acid, C₁-C₈-alkyl esters, chloroform, and/or inorganic solvents such as water, inorganic acids such as hydrochloric acid, and inorganic bases such as sodium hydroxide, and mixtures thereof.

Preferred solvents include ethanol, 1-propanol, isopropyl alcohol, ethyl acetate, butyl acetate, glycerol, propylene glycol, liquid polyethylene glycols etc. and mixtures thereof. Most preferred are ethanol, water and glycerol and mixtures thereof.

The plant extract of the specie Hedychium coronarium, as hereinbefore defined herein, has a concentration in a range of from 0.05% to 5.0% (weight/volume), more preferred of 0.1% to 3.0% (weight/volume), even more preferred of 0.2 to 2.0% (weight/volume).

Cosmetically acceptable auxiliary substances include:
pH-adjusting agents such as buffer substances;
Inorganic and organic acids or bases comprising all cosmetically acceptable inorganic and organic acids, which are well known to a person skilled in the art;
Fatty substances such as mineral oils, for example paraffin oils or Vaseline oils, silicone oils, and plant-derived oils, such as coconut oil, sweet almond oil, apricot oil, maize oil, jojoba oil, olive oil, avocado oil, sesame oil, palm oil, eucalyptus oil, rosemary oil, lavender oil, pine oil, thyme oil, mint oil, cardamom oil, orange blossom oil, soya oil, bran oil, rice oil, rapeseed oil and castor oil, wheat germ oil and vitamin E isolated there from, evening primrose oil; Animal oils or fats, such as tallow, lanolin, clarified butter, as well as neutral oil (Miglycol 812), squalane, fatty acid esters, esters of fatty alcohols, such as triglycerides, as well as the so-called basic cream (“Basiscreme”) DAC which is an important basic composition according to the German Codex for medicaments (Deutscher Arzneimittel Codex) comprising glycerolmonostearat, cetylalkohol, medium-chained triglycerides, white Vaseline, macrogol-1000-glycerolmonostearat, propyleneglycol, and purified water, and
Plant lecithin (e.g. soya lecithin), sphingolipids/ceramides isolated from plants,
Waxy substances having a melting point corresponding to skin temperature, such as beeswax, carnauba wax and candelilla, microcrystalline waxes, polyethylene or silicone waxes, and in particular all oils and waxes suitable for topical application, such as those mentioned, for example, in the CTFA publication Cosmetic Ingredient Handbook, 1st ed., 1988, The Cosmetic, Toiletry and Fragrance Association, Inc., Washington; surface-active agents, such as dispersing agents, wetting agents, emulsifiers etc.;
Fillers;
Stabilizers;
Co solvents;
Dyestuffs and pigments;
Preservatives;
Moisturizing agents;
Antioxidants;
UV-protecting agents
Softeners;
Lubricants or slip agents;
Skin-conditioning agents.

Preferred auxiliaries are selected from the groups of antioxidants, moisturizing agents, softeners, skin-conditioning agents, fat substances, such as in particular cosmetic fats and oils, and UV-protecting agents.

Generally, the classification of the abovementioned substances into the category of auxiliary substances in the context of the present invention does not exclude the fact that these auxiliary substances may also exhibit a certain activity, which applies in particular to a certain degree to coils and other auxiliaries with moisturizing and lipid replenishing effects.

The composition of the present invention may further comprise at least one additional cosmetically active agent. In the context of the invention, cosmetic agents or agents prepared using cosmetic active composition are essentially agents in the sense of the German Food and Feed Code [German=LFGB], i.e. substances or formulations of substances which are intended for external use on humans for skin care agents, cleansing, care, or for influencing the appearance or the body odour, or for imparting odoriferous impressions, unless they are predominantly intended for alleviation or elimination of diseases, suffering, body damage or pathological symptoms. Examples of cosmetically active agents generally include: ant acne agents, antimicrobial agents, antiperspirant agents, astringent agents, deodorizing agents, hair removal agents, conditioning agents for the skin, skin-smoothing agents, agents for increasing skin hydration, such as e.g. glycerol or urea, sunscreen agents, keratolytics, free radical scavengers for free radicals, antiseborrhoea agents, antidandruff agents, antiseptic active compounds, active compounds for treatment of signs of ageing of the skin and/or agents which modulate the differentiation and/or proliferation and/or pigmentation of the skin, vitamins, such as vitamin C (ascorbic acid) and its derivatives, such as, for example, glycosides, such as ascorbyl glucoside, or esters of ascorbic acid, such as sodium or magnesium ascorbyl phosphate or ascorbyl palmitate and stearate, L-ascorbic acid phosphate esters, alkali metal salts, such as sodium and potassium salts, of L-ascorbic acid phosphate esters; alkaline earth metal salts, such as magnesium and calcium salts, of L-ascorbic acid phosphate esters; trivalent metal salts, such as aluminium salts, of L-ascorbic acid phosphate esters; alkali metal salts of L-ascorbic acid sulfate esters, such as sodium and potassium salts of L-ascorbic acid sulfate esters; alkaline earth metal salts, such as magnesium and calcium salts, of L-ascorbic acid sulfate esters; trivalent metal salts, such as aluminium salts, of L-ascorbic acid sulfate esters; alkali metal salts, such as sodium and potassium salts, of L-ascorbic acid esters; alkaline earth metal salts, such as magnesium and calcium salts, of L-ascorbic acid esters; and trivalent metal salts, such as aluminium salts, of L-ascorbic acid esters, retinoids (retinol, retinal, retic acid), anthralins (dioxyanthranol), anthranoids, peroxides (in particular benzoyl peroxide), minoxidil, lithium salts, antimetabolites, vitamin D and its derivatives; catechols, flavonoids, ceramides, polyunsaturated fatty acids, essential fatty acids (e.g. gamma-linolenic acid), enzymes, coenzymes, enzyme inhibitors, hydrating agents, skin soothing agents, detergents or foam-forming agents, and inorganic or synthetic matting fillers, or decorative substances, such as pigments or dyestuffs and coloured particles for foundations, make-up formulations, and other agents for cosmetic adornment and coloured modelling of eyes, lips, face etc. and abrasive agents. Plant-derived active compound extracts or extracts or individual substances obtained there from (other than the Hedychium extract of the present invention) may furthermore be mentioned. Generally, the plant active compound extract may be selected from the group consisting of solid plant extracts, liquid plant extracts, hydrophilic plant extracts, lipophilic plant extracts, individual plant constituents; and mixtures thereof, such as flavonoids and their aglycan: rutin, quercetin, diosmin, hyperoside, (neo)hesperidin, hesperitin, Ginkgo biloba (e.g. ginkgoflavone glycosides), Crataegus extract (e.g. oligomeric procyanidins), buckwheat (e.g. rutin), Sophora japonica (e.g. rutin), birch leaves (e.g. quercetin glycosides, hyperoside and rutin), elder blossom (e.g. rutin), linden blossom (e.g. essential oil with quercetin and farnesol), St. John's wort oil (e.g. olive oil extract), Calendula, Arnica (e.g. oily extracts of the blossom with essential oil, polar extracts with flavonoids), Melissa (e.g. flavones, essential oil); immunostimulants: Echinacea purpurea (e.g. alcoholic extracts, fresh sap, pressed juice), Eleutherococcus senticosus; alkaloids: Rauwolfia (e.g. prajmalin), periwinkle (e.g. vincamin); further phytopharmaceuticals: Aloe, horse chestnut (e.g. aescin), garlic (e.g. garlic oil), pineapple (e.g. bromelains), ginseng (e.g. ginsenosides), Our Lady's thistle fruit (e.g. extract standardized with regard to silymarin), box holly root (e.g. ruscogenin), valerian (e.g. valepotriates, tct. Valerianae), kava (e.g. kava lactones), hop blossom (e.g. hop bitters), Passiflorae, gentian (e.g. ethanol. extract), anthraquinone-containing drug extracts, e.g. aloin-containing Aloe vera juice, pollen extract, algae extracts, liquorice extracts, palm extract, Galphimia (e.g. original tincture) mistletoe (e.g. aqueous ethanol. extract), phytosterols (e.g. beta-sitosterol), verbascum (e.g. aqueous alcohol. extract), Drosera (e.g. vinum liquorosum extract), sea buckthorn fruit (e.g. juice obtained there from or sea buckthorn oil), marshmallow root, primula root extract, fresh plant extracts of mallow, comfrey, ivy, horsetail, yarrow, ribwort (e.g. pressed juice), stinging nettle, greater celandine, parsley; plant extracts from Norolaena lobata, Tagetes lucida, Teeoma siems, Momordica charantia, and Aloe vera extracts.

Preferred cosmetic active compounds are those being active in the use according to the present invention, such as in particular skin care agents, skin conditioning agents, skin-smoothing agents, agents for increasing skin hydration, such as e.g. glycerol or urea, sunscreen agents, keratolytics, free radical scavengers against free radicals, antiseborrhoea agents, active compounds for treatment of signs of ageing of the skin and/or agents which modulate the differentiation and/or proliferation and/or pigmentation of the skin.

The cosmetic composition comprising the plant extract of the specie Hedychium coronarium extract according to the present invention is for topical application. Accordingly, Formulations suitable for topical application to skin and may be made into a wide variety of product types that include, without being limited thereto, lotions, creams, gels, emulsions, sticks, sprays, ointments, cleansing liquid washes and solid bars, shampoos, pastes, mousses, wipes, patches, cosmetic dressings or masks and adhesive bandages, hydrogels, and films or liposomal formulations.

The plant extract of the specie Hedychium coronarium is present in the composition according to the invention in an amount from about 0.001% to about 20% by weight, in particular in an amount from about 0.01% to about 10%, preferably in an amount from about 0.1% to about 5.0% by weight, more particularly in an amount from about 0.15% to about 3.0% by weight, even more preferably in an amount from about 0.2% to about 2.0% by weight, in each case based on the total weight of the composition.

The present invention further relates to a process for preparing a Hedychium coronarium roots and/or rhizomes extract according to claim 5, comprising the following steps:
A step a) which consists in providing a certain amount of pieces of roots and/or rhizomes of the Hedychium coronarium plant to be extracted;
A step b) during which said pieces of roots and/or rhizomes of the Hedychium coronarium plant are extracted with a suitable solvent or solvent mixture; to obtain an Hedychium coronarium plant roots and/or rhizomes pieces extraction mixture;
A step c) during which said Hedychium coronarium plant roots and/or rhizomes pieces extraction mixture obtained at step b), is filtrated to obtain a filtrate of Hedychium coronarium plant roots and/or rhizomes pieces extraction mixture;
A step d) during which said filtrate of Hedychium coronarium plant roots and/or rhizomes pieces extraction mixture obtained at step c), is let settle to separate the solid residues from the supernatant;
A step e) during which said supernatant obtained at step d) is filtrated to obtain a Hedychium coronarium plant roots and/or rhizomes extract as a clear solution;
A step f) during which said clear solution obtained at step e) is adjusted to the desired concentration using a suitable solvent or solvent mixture, to obtain the desired Hedychium coronarium roots and/or rhizomes extract.

In the process as hereinbefore defined, in step a) the pieces of plant extract of the specie Hedychium coronarium are preferably selected from crushed, cut or milled roots and/or rhizomes; in step b) the solvent or solvent mixture is preferably a hydrophilic or water-miscible solvent, such as preferably selected from the solvents as described above.

Preferably ethanol is used for extraction of the Hedychium pieces in step b). Preferably one or more hydroalcoholic extractions (preferably using ethanol) are carried out. Preferably the extraction is carried out using the plant pieces and hydroalcoholic solvent in a ratio 1/10 (e.g. 1 kg of plant pieces, such as in particular root/rhizome pieces extracted with 10 dm³ of 50% (w/w) ethanol). In step c) conventional filtration techniques are applied, which are well known to a person skilled in the art; In step d), the separation of the solid residues and the supernatant is carried out using conventional techniques, which are well known to a person skilled in the art; In step e), the filtration of the supernatant is carried out for further clarification and purification of the supernatant to obtain the Hedychium extract in the form of a clear solution; In step f), the adjustment of the clear solution to the desired concentration, is achieved with a suitable solvent or solvent mixture preferably selected from the solvents as described above and more preferably with a mixture of water and glycerol in a volume ratio from 10/90 to 90/10, preferably from 20/80 to 80/20.

According to a specific embodiment, in step f) of the process as hereinbefore defined, the adjustment of the clear solution to the desired concentration, is achieved with water/glycerol mixture of which ratio (volume/volume) is equal to 30/70.

The Hedychium coronarium roots and/or rhizomes extracts obtained according to the process that is the subject of the invention can be introduced into a further formulation which can be administered orally, topically or parenterally.

The formulation for topical use is characterised in that it comprises at least one cosmetically acceptable excipient and an effective quantity of the Hedychium coronarium roots and/or rhizomes extracts obtained according to the process that is the subject of the invention.

The expression “for topical use” used in the definition of the formulation as described above means that said formulation is used by application on the skin, whether it be a case of a direct application or an indirect application for example in the case of a body care product in the form of a textile or paper wipe or sanitary products intended to be in contact with the skin.

The expression “cosmetically acceptable” used in the definition of the formulation for topical use as described above means, according to the directive of the Council of the European Economic Community No 76/768/CEE of 27 Jul. 1976 as amended by directive No 93/35/CEE of 14 Jun. 1993, that the formulation comprises any substance or preparation intended to be put in contact with the various parts of the human body (epidermis, hair or pilous system, nails, lips and genital organs) or with the teeth and the mouth mucosa with a view, solely and mainly, to cleansing them, to perfuming them, to modifying the appearance thereof and/or to correcting body odours thereof and/or to protecting or keeping them in good condition.

The formulation for topical use comprising at least one cosmetically acceptable excipient and an effective quantity of the Hedychium coronarium roots and/or rhizomes extracts obtained according to the process that is the subject of the invention, is generally in the form of dilute aqueous or water/alcohol solutions, in the form of single or multiple emulsions, such as water in oil (W/O), oil in water (O/W) or water in oil in water (W/O/W) emulsions, in which the oil is of a plant or mineral nature, or in powder form. They may also be dispersed or impregnated on textile or on non-woven materials, whether it be wipes, paper towels or garments.

In general terms, the Hedychium coronarium roots and/or rhizomes extracts obtained according to the process that is the subject of the invention, is associated with numerous types of adjuvants or active ingredients used in the topical formulations as defined above, whether it be a case of fats, organic solvents, thickeners, gelling agents, softeners, foaming surfactants and/or detergents, superfatting agents, thickening and/or gelling surfactants, antioxidants, opacifiers, stabilisers, foaming agents, perfumes, emulsifying surfactants, hydrotropic agents, plasticers, superfatting agents, texture agents, pigments, sequestring agents, chelating agents, preservatives, essential oils, dyes, hydrophilic or lipophilic active agents, moisteners, perfumes, mineral or organic sun filters, mineral fillers, or any other ingredient normally used in cosmetics.

Examples of oils that can be associated with the Hedychium coronarium roots and/or rhizomes extracts obtained according to the process that is the subject of the invention, in the formulations for topical use, include mineral oils such as paraffin oil, vaseline oil, isoparaffins or mineral white oils, oils of animal origin such as squalene or squalane, vegetable oils such as sweet almond oil, coprah oil, castor oil, jojoba oil, olive oil, rapeseed oil, ground nut oil, sunflower oil, wheatgerm oil, maize germ oil, soya oil, cotton oil, alfalfa oil, poppy oil, pumpkin oil, evening primrose oil, millet oil, barley oil, rye oil, safflower oil, candleberry oil, passion flower oil, hazelnut oil, palm oil, shea butter, apricot kernel oil, calophyllum oil, sysymbrium oil, avocado oil, calendula oil; ethoxylated plant oils; synthetic oils such as fatty acid esters such as butyl myristate, propyl myristate, cetyl myristate, isopropyl palmitate, butyle stearate, hexadecyl stearate, isopropyl stearate, octyl stearate, isocetyl stearate, dodecyl oleate, hexyl laurate, propyleneglycol dicaprylate, ester derivatives of lanolic acid, such as isopropyl lanolate, isocetyl lanolate, monglycerides, diglycerides and triglycerides of fatty acids such as glycerol triheptonoate, alkylbenzoates, polyalfaolenfins, polyolefins such as polyisobutene, synthesis isoalkane, such as isohexadecane, isododecane, perfluorinated oils and silicone oils. The latter include more particularly dimethylpolysiloxanes, methylphenylpolysiloxanes, silicones modified by amines, silicones modified by fatty acids, silicones modified by alcohols, silicones modified by alcohols and fatty acids, silicones modified by polyether groups, modified epoxy silicones, silicones modified by fluorinated groups, cyclic silicones and silicones modified by alkyl groups.

Other fats that can be associated with the Hedychium coronarium roots and/or rhizomes extracts obtained according to the process that is the subject of the invention, in the formulations for topical use, include fatty alcohols and fatty acids.

Examples of waxes that can be associated with the Hedychium coronarium roots and/or rhizomes extracts obtained according to the process that is the subject of the invention, in the formulations for topical use, include beeswax; carnauba wax; candelilla wax; ouricoury wax; Japan wax; cork fibre wax or sugarcane wax; paraffin waxes, lignite waxes; microcristalline waxes; lanolin wax; ozocerite; polyethylene wax; hydrogenated oils; silicone waxes; vegetable waxes; fatty alcohols and fatty acids solid at ambient temperature; glycerides solid at ambient temperature.

Examples of thickening and/or emulsifying polymers that can be associated with the Hedychium coronarium roots and/or rhizomes extracts obtained according to the process that is the subject of the invention, in the formulations for topical use, include homopolymers, or copolymers of acrylic acid or derivatives of acrylic acid, homopolymers or copolymers of acrylamide, homopolymers or copolymers of acrylamide derivatives, homopolymers or copolymers of acrylamido methylpropane sulfonic acid, vinyl monomer, trimethylaminoethyl acrylate chloride, hydrocolloids of plant or biosynthetic origin, for example xanthan gum, karaya gum, carraghenates, alginates; silicates; cellulose and derivatives thereof; starch and hydrophylic derivatives thereof; polyurethanes.

Polymers of the polyelectrolyte type that can be associated with the Hedychium coronarium roots and/or rhizomes extracts obtained according to the process that is the subject of the invention, in the formulations for topical use, include for example copolymers of acrylic acid and 2-methyl-[(1-oxo-2-propenyl)amino] 1-propane sulfonic acid (MPSA), copolymers of acrylamine and 2-methyl-[(1-oxo-2-propenyl)amino] 1-propane sulfonic acid, copolymers of 2-methyl-[(1-oxo-2-propenyl)amino] 1-propane sulfonic acid and (2-hydroxyethyl) acrylate, the homopolymer of 2-methyl-[(1-oxo-2-propenyl)amino] 1-propane sulfonic acid, the homopolymer of acrylic acid, the copolymers of acryloyl ethyl trimethyl ammonium chloride and acrylamide, the copolymers of MPSA and vinylpyrolidone, the copolymers of acrylic acid and alkyl acrylates the carbon chain of which comprises between ten and thirty carbon atoms, the copolymers of MPSA and alkyl acrylates the carbon chain of which comprises between ten and thirty carbon atoms. Such polymers are sold respectively under the names SIMULGEL™ EG, SEPIGEL™ 305, SIMULGEL™ NS, SIMULGEL™ INS 100, SIMULGEL™ FL, SIMULGEL™800, SIMULGEL™ A by the applicant.

Examples of emulsifiers that can be associated with the Hedychium coronarium roots and/or rhizomes extracts obtained according to the process that is the subject of the invention, in the formulations for topical use, include fatty acid salts, ethyloxated fatty acids, esters of fatty acid and sorbitol, esters of ethyloxated fatty acids, polysorbates, polyglycerol esters, ethyloxated fatty alcohols, sucrose esters, alkylpolyglycosides, sulfated and phosphated fatty alcohols or the mixtures of alkylpolyglycosides and fatty alcohols described in the French patent applications 2 668 080, 2 734 496, 2 756 195, 2 762 317, 2 784 680, 2 784 904, 2 791 565, 2 790 977, 2 807 435 and 2 804 432.

Examples of foaming surfactants and/or detergents that can be associated with the Hedychium coronarium roots and/or rhizomes extracts obtained according to the process that is the subject of the invention, in the formulations for topical use, include the topically acceptable anionic, cationic, amphoteric or non-ionic surfactants normally used in this field of activity.

The anionic surfactants that can be associated with the Hedychium coronarium roots and/or rhizomes extracts obtained according to the process that is the subject of the invention, in the formulations for topical use, include particularly alkaline metal salts, alkaline earth metal salts, ammonium salts, amine salts, the aminoalcohol salts of the following compounds: alkylether sulfates, alkyl sulfates, alkylamidoether sulfates, alkylarylpolyether sulfates, monoglyderide sulfates, alpha-olefin sulfates, paraffin sulfonates, alkyl phosphates, alkylether phosphates, alkyl sulfonates, alkylamide sulfonates, alkylaryl sulfonates, alkylcarboxylates, alkylsulfosuccinates, alkylether sulfosuccinates, alkylamidesulfosuccinates, alkylsulfoacetates, alkylsarcosinates, acylated thionates, N-acyltaurates and acyllactates.

The anionic surfactants that can be associated with the Hedychium coronarium roots and/or rhizomes extracts obtained according to the process that is the subject of the invention, in the formulations for topical use, also include the N-acylated derivatives of amino acids, peptides, proteins the acyl chain of which comprises 8 to 16 carbon atoms; fatty acid salts, acid salts of coprah oil, optionally hydrogenated.

The amphoteric surfactants that can be associated with the Hedychium coronarium roots and/or rhizomes extracts obtained according to the process that is the subject of the invention, in the formulations for topical use, include particularly alkybetaines, alkylamidobetaines, sultaines, alkylamidoalkylsulfobetaines, imidazoline derivaties, phosphobetaines, amphopolyacetates and amphoproprionates.

The cationic surfactants that can be associated with the Hedychium coronarium roots and/or rhizomes extracts obtained according to the process that is the subject of the invention, in the formulations for topical use, include particularly the quaternary ammonium derivatives.

The non-ionic surfactants that can be associated with the Hedychium coronarium roots and/or rhizomes extracts obtained according to the process that is the subject of the invention, in the formulations for topical use, include particularly the alkylpolyglycosides the alkyle chain of which comprises 8 to 16 carbon atoms, castor oil derivatives, polysorbates, coprah amides, N-alkylamines and amine oxides.

Examples of texture agents that can be associated with the Hedychium coronarium roots and/or rhizomes extracts obtained according to the process that is the subject of the invention, in the formulations for topical use, include N-acylated derivatives of amino acids, such as for example the lauroyl lysine sold under the name AMINOHOPE™ LL by the company AJINOMOTO, the octenyl starch succinates sold under the name DRYFLO™ by the company NATIONAL STARCH, the myristyl polyglucoside sold by SEPPIC under the name MONTANOV 14, cellulose fibres, cotton fibres, chitosan fibres, talc, sericite or mica. Examples of opacifiers and/or pearling agents that can be associated with the Hedychium coronarium roots and/or rhizomes extracts obtained according to the process that is the subject of the invention, in the formulations for topical use, include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols.

Examples of thickening and/or gelling surfactants that can be associated with the Hedychium coronarium roots and/or rhizomes extracts obtained according to the process that is the subject of the invention, in the formulations for topical use, include:
fatty esters of alkylpolyglycosides, optionally alkoxylated, and especially ethoxylated methylpolyglucoside esters such as PEG 120 methyl glucose trioleate and PEG 120 methyl glucose dioleate sold respectively under the names GLUCAMATE™ LT and GLUMATE™ DOE120;
alkoxylated fatty esters such as PEG 150 pentaerythrytyl tetrastearate sold under the name CROTHIX™ DS53, PEG 55 propylene glycol oleate sold under the name ANTIL™ 141;
fatty-chain polyalkylene glycol carbamates such as PPG 14 laureth isophoryl dicarbamate sold under the name EFLACOS™ T211, PPG 14 palmeth 60 hexyl dicarbamate sold under the name ELFACOS™ GT2125.

Examples of sun filters that can be associated with the Hedychium coronarium roots and/or rhizomes extracts obtained according to the process that is the subject of the invention, in the formulations for topical use, include all those appearing in the amended cosmetic directive 76/768/EEC appendix VII.

Examples of active ingredients that can associated with the Hedychium coronarium roots and/or rhizomes extracts obtained according to the process that is the subject of the invention, in the formulations for topical use, include the compounds having a lightening or depigmenting action such as for example arbutin, kojic acid, hydroquinone, ellagic acid, vitamin C, magnesium ascorbyl phosphate, extracts of polyphenols, derivatives of glycosylated polyphenols such as Rosmarinyl glucoside, grape extracts, pine extracts, wine extracts, extracts of olives, pond extracts, N-acylated proteins, N-acylated peptides, N-acylated amino acids, partial hydrolysates of N-acylated proteins, amino acids, peptides, total hydrolysates of proteins, partial hydrolysates of proteins, polyols (for example glycerine or butylene glycol), urea, pyrrolidone carboxylic acid or derivatives of this acid, glycyrrhetinic acid, alpha-bisabolol, sugars or derivatives of sugars, polysaccharides or derives thereof, hydroxyacids, for example lactic acid, vitamins, vitamin derivatives such as Retinol, vitamin E and derivatives thereof, minerals, enzymes, co-enzymes such as co-enzyme Q10, hormones or hormone-like substances, soya extracts, for example Raffermine™, wheat extracts, for example Tensine™ or Gliadine™, plant extracts such as tanin-rich extracts, isoflavone-rich extracts or terpene-rich extracts, extracts of fresh or seawater algae, essential waxes, bacterial extracts, minerals, lipids in general, lipids such as ceramids or phospholipids, active agents having a slimming action such as caffeine or derivatives thereof, such as quinoa extracts sold under the name ADI POLESS™, such as the Canadian hemlock extract sold under the name SERENIKS™ 207, such as the composition comprising Lauroyl Proline sold under the name ADIPOSLIM™, the active agents having an antimicrobial activity or purifying action vis-à-vis oily skins such as LIPACIDE™ PVB, active agents having an energising or stimulating property such as SEPITONIC™ M3 or Physiogenyl™, panthenol and derivatives thereof such as SEPICAP™ MP, anti-aging active agents such as SEPILIFT™ DPHP, LIPICIDE™ PVB, SEPIVINOL™, SEPIVITAL^{T}M hydrating active agents such as SEPICALM™ S, SEPICALM™ VG and SEPILIFT™ DPHP, “anti-photo aging” anti-aging active agents, active agents protecting the integrity of the dermo-epidermal junction, active agents increasing the synthesis of components of the extracellular matrix, active agents having a slimming activity such as caffeine, theophylline, AMPc, green tea, sage, ginko biloba, ivy, horse chesnut, bamboo, ruscus, butcher's broom, centella asiatica, heather, ulmaria, fucus, rosemary, willow, active agents creating a “heating” sensation on the skin such as skin microcirculation activators (for example nicotinates) or products creating a sensation of “coolness” on the skin (for example menthol and derivatives thereof).

The invention is further illustrated by the following examples, which relate to certain specific embodiments of the present invention. The examples were carried out using well known standard techniques within the routine to those of skill in the art, unless indicated otherwise. The following examples are for illustrative purposes only and do not purport to be wholly definitive as to conditions or scope of the invention. As such, they should not be construed in any way as limiting the scope of the present invention.

FIG. 1 shows the results regarding mitochondrial protection of Example 2 with ATP titration in fibroblasts treated for 48 h with HCR extract (with irradiation or not).

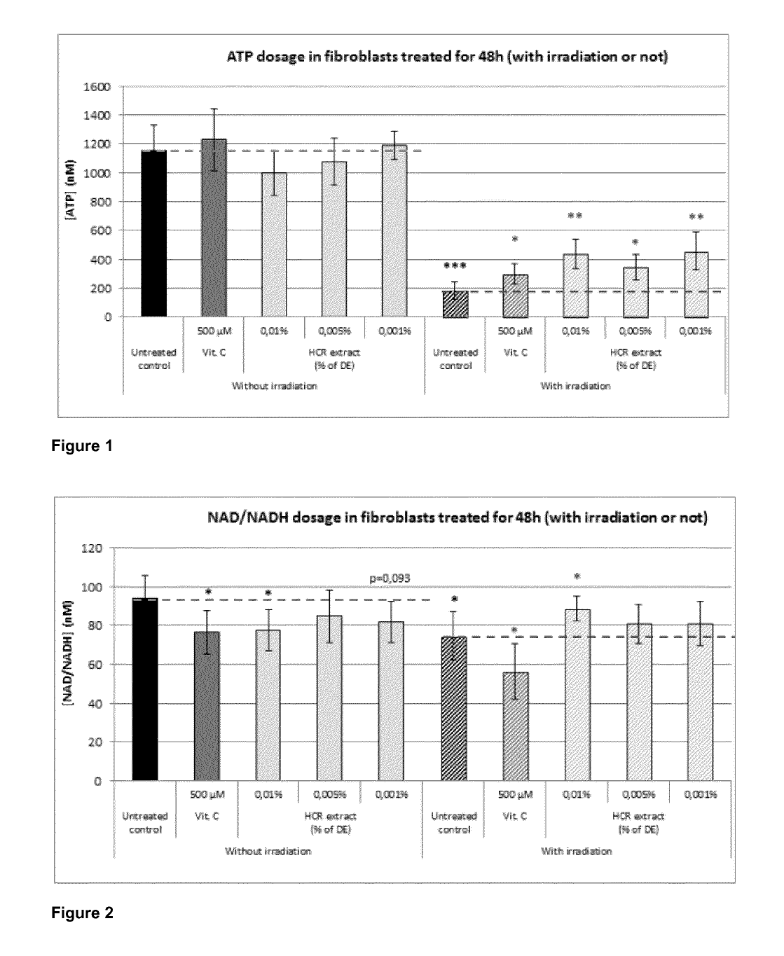

FIG. 2 shows the results regarding mitochondrial protection of Example 2 with NAD⁺/NADH titration in fibroblasts treated for 48 h with HCR extract (with irradiation or not).

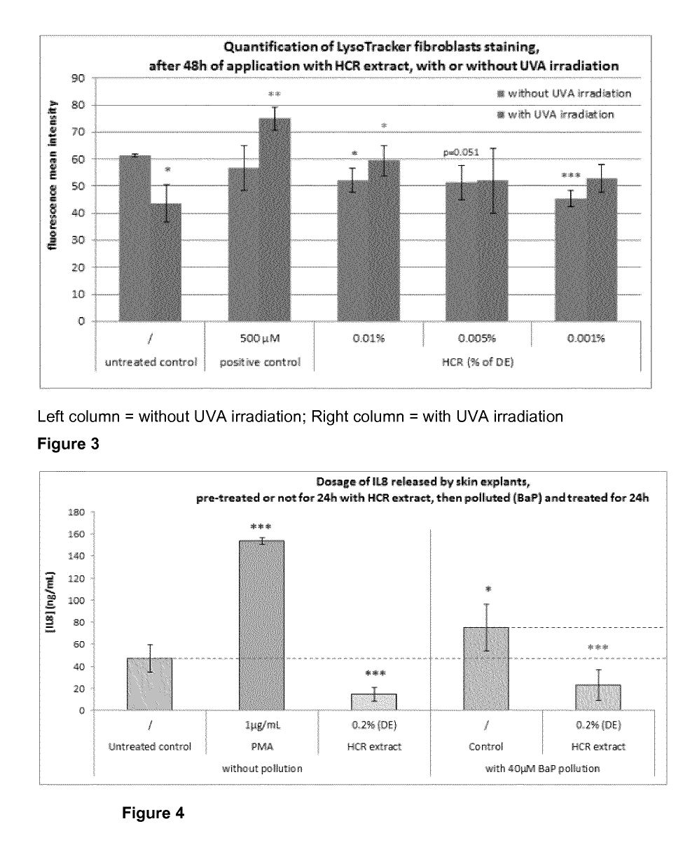

FIG. 3 shows the results regarding lysosomal protection of Example 3 with quantification of LysoTracker fibroblasts staining after treatment for 48 h with HCR extract (with irradiation or not).

FIG. 4 shows the results regarding IL-8 released by skin explants, pre-treated or not for 24 h with HCR extract, polluted with BaP and re-treated for 24 h of Example 4.

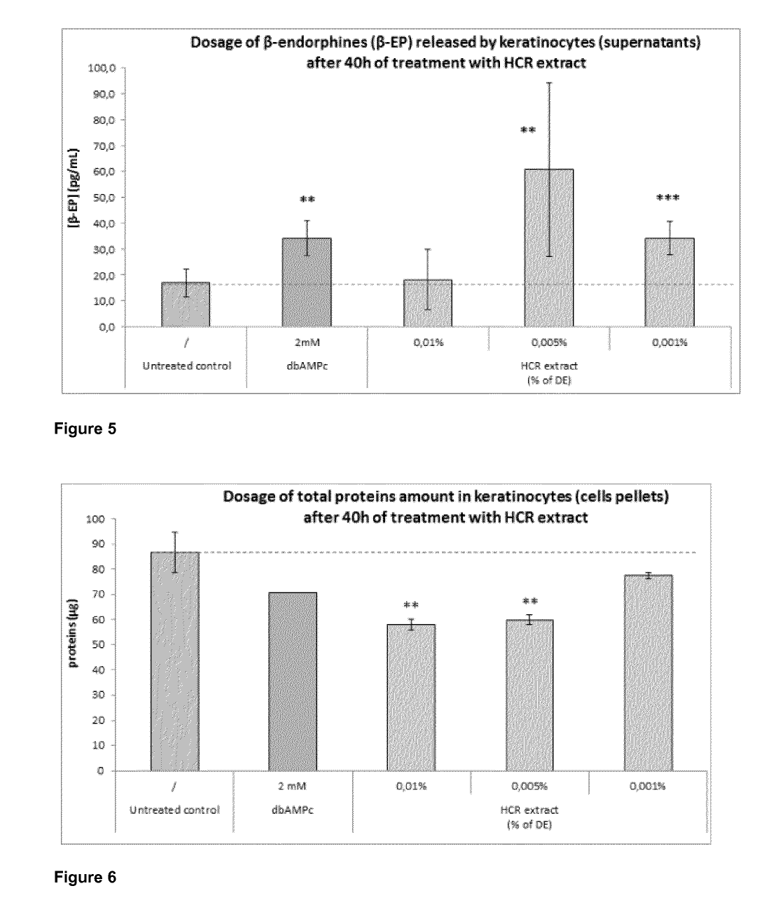

FIG. 5 shows the results regarding titration of β-endorphins released by keratinocytes after treatment for 40 h with HCR extract of Example 5.

FIG. 6 shows the results regarding titration of total proteins amount in keratinocytes after treatment for 40 h with HCR extract of Example 5.

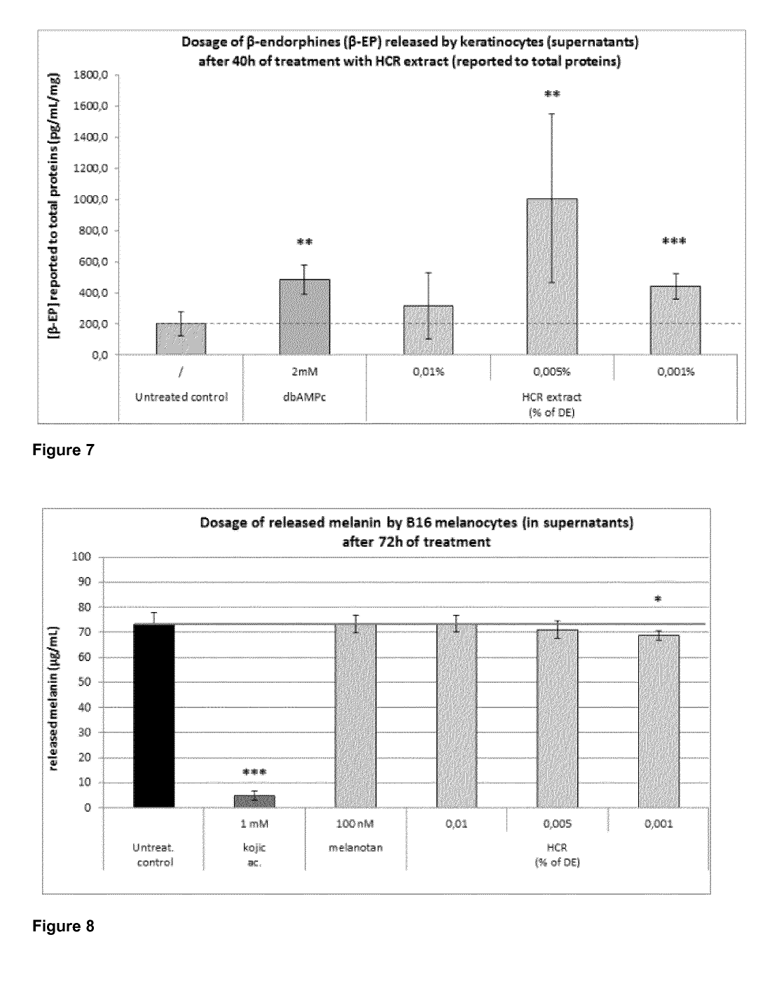

FIG. 7 shows the results regarding titration of β-endorphins released by keratinocytes after treatment for 40 h with HCR extract reported to total protein of Example 5.

FIG. 8 shows the results regarding titration of released melanin by B16 melanocytes after treatment for 72 h with HCR extract concentrations 0.001, 0.005 and 0.01% of DE of Example 6.

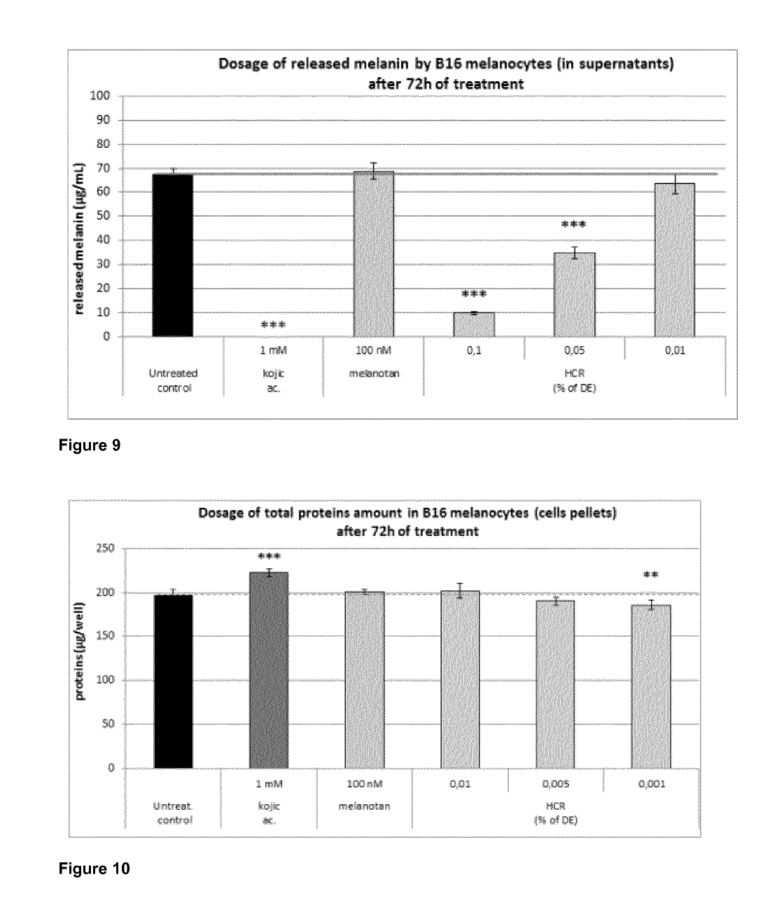

FIG. 9 shows the results regarding titration of released melanin by B16 melanocytes after treatment for 72 h with HCR extract concentrations 0.01, 0.05 and 0.1% of DE of Example 6.

FIG. 10 shows the results regarding titration of total proteins amount in B16 melanocytes after treatment for 72 h with HCR extract concentrations 0.001, 0.005 and 0.01% of DE of Example 6.

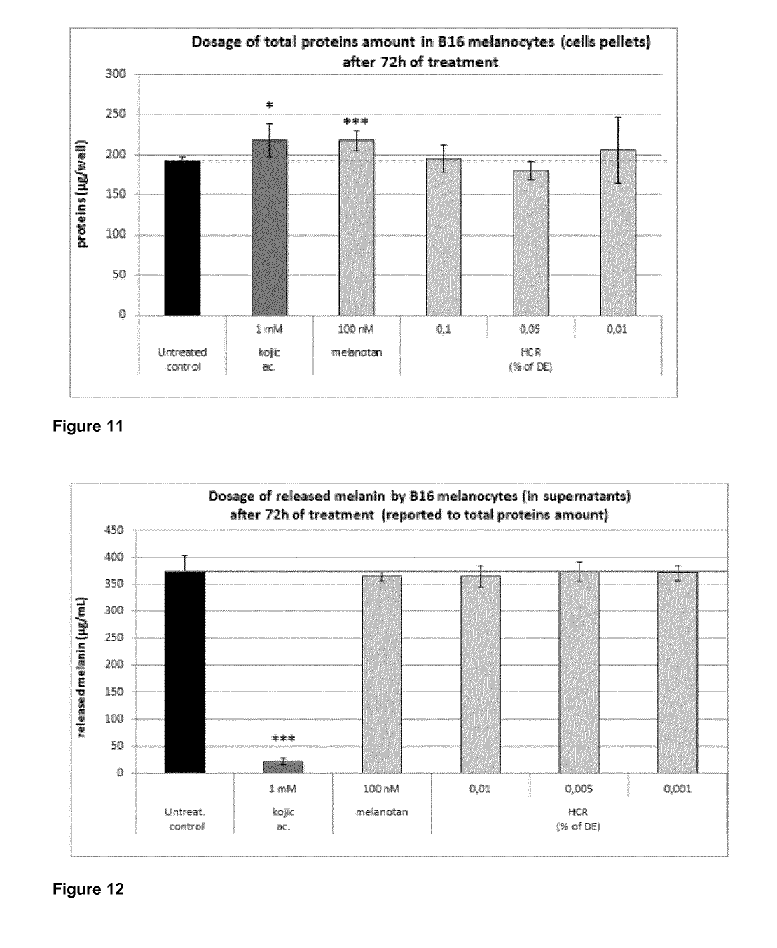

FIG. 11 shows the results regarding titration of total proteins amount in B16 melanocytes after treatment during seventy-two hours with HCR extract concentrations 0.01%, 0.05% and 0.1% of DE of Example 6.

FIG. 12 shows the results regarding titration of released melanin by B16 melanocytes after the treatment during seventy-two hours with HCR extract concentrations 0.001%, 0.005% and 0.01% of DE reported to total protein of Example 6.

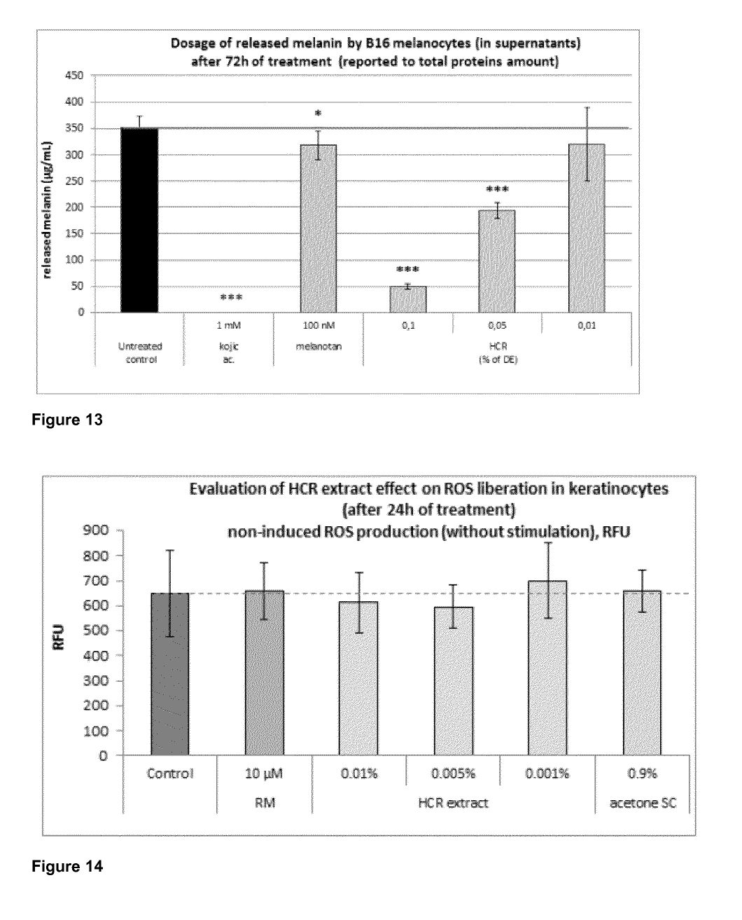

FIG. 13 shows the results regarding titration of released melanin by B16 melanocytes after treatment during seventy-two hours with HCR extract concentrations 0.01% 0.05% and 0.1% of DE reported to total protein of Example 6.

FIG. 14 shows the results regarding the effects of HCR extract on ROS liberation in keratinocytes after twenty-four hours of treatment without ROS production stimulation of Example 7.

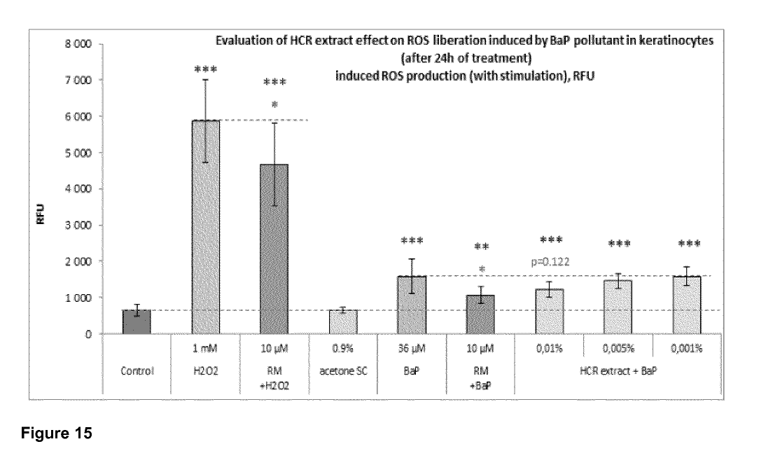

FIG. 15 shows the results regarding the effects of HCR extract on ROS liberation in keratinocytes after twenty-four hours of treatment with ROS production stimulation of Example 7.

### EXAMPLE 1

### Preparation of a Hedychium coronarium Root Extract

An Hedychium coronarium root extract is prepared according to the process of the present invention.

In step (a), crushed roots (rhizomes) of Hedychium coronarium, having a size of up to 10 cm length, are provided in a suitable extraction vessel.

In step (b), a mixture water/ethanol [50/50 (w/w)] is added as the extraction solvent to the crushed roots and 2 extractions are carried out.

In step (c), the extraction mixture is filtered using conventional filtering techniques.

In step (d), the filtrate of step (c) is concentrated by settling of the solid residues, followed by decantation of the supernatant.

In step (e), the supernatant of step (d) is filtered using conventional filtering techniques.

In step (f), adjustment of the clear solution of step (e) is carried out to a solution of 20 g/l in a mixture of water and glycerol having the ratio of 30/70 (v/v), followed by a final filtration for further purification to obtain the final extract in the desired concentration of 2.0% (w/v).

### EXAMPLE 2

### Evaluation of the Effects of Hedychium coronarium Root Extract (HCR Extract) on Mitochondrial Network in Fibroblasts Irradiated with UVA (MitoTracker Staining, ATP and NAD+/NADH Titrations)

### 1)—Background Information

a)—The aim of this study was to evaluate a HCR extract effects on mitochondria distribution and cell energy metabolism of UVA-irradiated fibroblasts.

b)—Normal human fibroblasts (NHF) extracted after plastic surgery from mammary skin dermis of a forty-one year-old donor (Caucasian woman), were cultured as primary monolayers;

c)—The test extract was then applied during twenty-four hours,

d)—Cells were irradiated (without treatment) and the test compound was applied again during another twenty-four hours;

e)—Mitochondria were stained, or cells were collected to perform ATP (adenosine triphosphate) and NAD⁺/NADH (nicotinamide adenine dinucleotide) titrations.

### 2)—Dose Determination (Cytotoxicity/Viability Check by XTT Assay) and Evaluated Test Compound

a)—After evaluation of the working doses by checking cell viability/toxicity with XTT assay a test concentration of 2% HCR dry extract (corresponding to a dilution of 1/20 HCR in the culture medium to finally obtain 0.1% HCR) was selected.

b)—Cells were seeded into 96-wells microplates (at a cell density of 5,000 cells per well, i.e. 15,000 NHF/cm², the density used later for titrations), 24 hours before treatment, either in Dulbecco's modified Eagle medium or in DMEM (Gibco™ 41966), with antibiotics (Gibco™ 15140) and 10% of foetal bovine serum (FBS) (Gibco™ 10270) (incubation at 37° C.; 5% CO₂). Treatments were done in medium without FBS (48 hours; 37° C.; 5% CO₂).

c)—After 48 hours of treatment, wells were emptied and gently rinsed with PBS (Phosphate Buffer Saline). A XTT solution (0.3 mg/ml) was then applied on cells and the plate was then incubated in the dark (3 hours, 37° C.; 5% CO₂). After three hours of incubation, the absorbance was measured at 450 nm with 650 nm reference. Blanks were also achieved, with XTT solution in wells without cells (but with product or not), in order to check the absence of interaction between the XTT molecule and the extract, that would be a bias in the results. The XTT test was performed using the kit “Cell Proliferation Kit II (XTT)” (Roche Diagnostics™ 11465015001). The XTT system, a colorimetric method, is an assay to quantify mitochondrial activity and was used as a viability test. This test is based on the cleavage of yellow tetrazolium salt XTT to orange formazan, by the system “succinate-tetrazolium reductase” present in the mitochondrial respiratory chain of cells. Thus, this conversion only occurs only in metabolically active cells, i.e. living cells. The derivative formazan is measured by spectrophotometry at 450 nm with 650 nm reference.

d)—Means of optic density data (OD, or absorbance) were calculated in each case.

The viability of treated cells is expressed as percentage of untreated control:

$$\% {viability}_{{sample}^{″}} = \frac{{OD}_{{ }^{″} {sample}^{″}}}{OD {Mean}_{{ }^{″} untreated {control}^{″}}} \times 100$$

A treatment that diminishes cells viability, below the 80% limit of mitochondrial activity compared to untreated control, was considered as toxic for the cells. On the contrary, an increase of data detects mitochondrial activity.

### 3)—Evaluation of Mitochondrial Network Protection

a)—Fibroblasts were seeded into Petri dishes (at a cell density of 150,000 cells per dish, with a diameter of 60 mm, i.e. 7,700 NHF/cm²), in the same medium as previously described (complete medium, i.e. with FBS), one day before treatment (incubation at 37° C.; 5% CO₂). Each test compound concentration/dose (in one dish each) was then applied for 24 hours (in medium without sera; incubation at 37° C./5% CO₂). Then cells were UVA-irradiated or not in a saline buffer (8 J/cm², in a Vilber Lourmat RMX 3W™ machine) and finally treated again with the test compound (in culture media) for another 24 hours.

b)—To evaluate the mitochondria staining, cells were incubated with a fluorescent probe (Invitrogen MitoTracker™). Cells were observed and photos were taken with ×40 objective (Olympus CK40™ microscope and Archimed Microvision software), with or without appropriate fluorescent filter. The photos allowed the visualization of the mitochondria network or of the whole cells, respectively.

### 4)—Evaluation of the Effects on the Metabolic Activity (ATP & NAD⁺/NADH Titrations)

a)—In order to check the effects of UV and the tested product on cells metabolism, ATP and NAD⁺/NADH titrations were performed.
(i)—Cells were seeded into 96-wells plates (at a cell density of 5,000 cells per well, i.e. 15,000 NHF/cm²), in the same medium as previously described, one day before treatment. Then the treatment and irradiation timing was the same: first application for twenty-four hours (in medium without sera), then UVA-irradiation or not (same dose) and finally a second treatment for another twenty-four hours. As formerly, from the beginning to the end of the experiment, cells were incubated at 37° C./5% CO₂. Here each dose was tested in quintuplicate (for ATP) or in sextuplicate (for NAD⁺/NADH).
(ii)—In order to proceed to the titrations, cells lysates or pellets were collected at the end of treatment. For titration of cellular ATP total level, detergent was added in wells containing cells and supernatants (media with product) to lyse cells and obtain cells lysates. For intracellular NAD⁺ and NADH titration, supernatants were eliminated and cells were made permeable to obtain permeable cells pellets.
(iii)—Then titrations were performed with commercial kits: the luminescent ATP detection assay kit (Abcam™ ab113849) and the NAD⁺/NADH cell-based assay kit (Cayman Chemical™ 600480).
(iv)—The NAD assay kit doses the two existing forms (oxidized NAD⁺ and reduced NADH). Firstly, adding of ethanol and alcohol dehydrogenase permits to reduce NAD+ found in samples to NADH, concomitantly to oxidation of ethanol. Then total NADH is oxidized in the same time as the reduction of a tetrazolium salt substrate (WST-1) to a coloured formazan. Measured quantity of this product (absorbance) is proportional to total NAD (NAD⁺ and NADH) in each sample.
(v)—The ATP assay kit is an ATP monitoring system based on firefly (Photinus pyralis) luciferase. Added luciferase and D-luciferin react with ATP in samples to produce light, which measure (luminescence) is proportional to the ATP concentration.

b)—Raw data of both ATP and NAD⁺/NADH titrations, i.e. luminescence units (RLU) or OD measurements obtained for respective standards, were plotted on graphics to determine standard curves. Then, the concentrations of ATP or NAD⁺/NADH measured in the samples were determined. Quantitative values of each condition were averaged. Data were graphically presented as concentrations (nM for ATP or NAD⁺/NADH). The results obtained for each condition can also be expressed relatively to the untreated control, with or without irradiation (set to 100%), or to the respective same condition without irradiation (evaluation of UVA effect):

%_{«sample»}=(Mean RLU or OD_{«sample»}Mean RLU or OD_{«control irradiated or not»})×100

The statistically significant effects of the results were determined by the Student's t-test using the same criteria as described for the XTT assay (comparison to non-irradiated or irradiated control, or to the respective same condition without irradiation).

### 5)—Results

a)—Dose Determination (Cytotoxicity/Viability Check by XTT Assay)

No cytotoxicity was observed for the tested concentrations between 0.0005% and 0.01% HCR (dry extract (DE)) on fibroblasts:

| | Concentrations to test |
|---|---|
| | MitoTracker assay and metabolism dosages) |
| HCR | 0.01-0.005-0.001% (of DE) |

b)—Evaluation of Mitochondrial Network Protection

Observation of the images allowed observing the effect of UV irradiation on cells shape and mitochondria distribution in cells. Indeed, some UV-irradiated fibroblasts were no more spindle-shaped and mitochondria network seemed less organized than without UV irradiation. With application of the positive control (vitamin C, 500 μM), effects of UVA were less distinct. Treatment of fibroblasts with HCR at 0.01% (of DE) protected mitochondria network impaired by UV irradiation. This effect against UV has not been observed with the lower doses of 0.005% and 0.001%.

c)—Evaluation of Effect on Metabolic Activity (ATP & NAD⁺/NADH Titrations)

Results of titrations are presented in FIG. 1. Data were expressed as concentrations (nM) reported to non-irradiated or irradiated control, or non-irradiated respective condition. Statistics p-values are calculated in comparison to non-irradiated control or irradiated control. Without irradiation, none of the treatments induced significant inhibition or stimulation of ATP production. UV irradiation induced a strong and very highly significant inhibition of ATP production by fibroblasts (−84%***, irradiated control compared to basis level, i.e. without irradiation). The positive reference (vitamin C, 500 μM) allowed significantly counteracting the UVA effect on this energy synthesis (+61%* compared to irradiated control). With HCR treatment, a significant or highly significant increase of ATP synthesis was observed: +136%**, +86%* and +146%** with, respectively, 0.01%, 0.005% and 0.001%.

d)—NAD⁺/NADH Titrations
(i)—In the absence of irradiation, all treatments tended toward inhibition of basis NAD⁺/NADH fibroblastic production. The results are shown in FIG. 2.
(ii)—With UVA irradiation, this coenzyme synthesis by NHF was significantly inhibited (−21%*, compared to basis level). Here Vitamin C did not show expected positive effect on NAD⁺/NADH release by UV-irradiated cells. HCR application at the highest tested dose (0.01%) allowed restoring NAD⁺/NADH fibroblastic production decreased by UVA (+19%*, compared to irradiated control). At the two other tested doses, the effect was not significant (+9%).

e)—The present evaluation hence shows that UVA induces a partial deconstruction/disintegration of mitochondria network and an inhibition of ATP and NAD⁺/NADH productions. The HCR extract shows effects on cultured and UV-irradiated fibroblasts, significantly increasing the ATP production and stimulating the NAD+/NADH synthesis. It allows protecting the mitochondria network against UV irradiation. Accordingly, the HCR extract is efficient to protect skin against UV damage on mitochondria and energy metabolism.

### EXAMPLE 3

### Evaluation of the Effects of HCR Extracts on Lysosomal Network in Fibroblasts Irradiated with UVA (LysoTracker Staining)

### 1)—Background Information

a)—The aim of this study was to evaluate HCR extract effects on lysosomes distribution in UVA-irradiated fibroblasts.

b)—Normal human fibroblasts (NHF) extracted after plastic surgery from mammary skin dermis of a forty-one year-old donor (Caucasian woman), were cultured as primary monolayers;

c)—The test extract was then applied during twenty-four hours,

d)—Cells were irradiated (without treatment) and the test compound was applied again during another twenty-four hours;

e)—Then the lysosomes were stained with a specific test compound.

### 2)—Dose Determination (Cytotoxicity/Viability Check by XTT Assay) and Evaluated Test Compound

The dose determination with XTT assay was carried out as described in Example 2.

### 3)—Evaluation of Lysosomes Network Protection

a)—Fibroblasts seeding was carried out as described in Example 2.

b)—Cells were then incubated with a fluorescent probe (Invitrogen LysoTracker™). They were observed and photographed with ×40 objective (Olympus CK40 microscope and Archimed Microvision software), with or without appropriate fluorescent filter. The photos allowed the visualization respectively of the lysosomes network or of the whole cells.

c)—Fluorescence was quantified with Colombus software, by HCS Pharma (Rennes, France), as mean intensity (3 images for each condition). It is the mean value of intensity for each pixel of staining, contained in the cell area stained with LysoTracker (number of cells of each image is taken into account, as the final value is the mean of all cells values on the image).

### 4)—Results

a)—Dose Determination (Cytotoxicity/Viability Check by XTT Assay)

No cytotoxicity was observed for the tested concentrations between 0.0005% and 0.01% HCR (DE) on fibroblasts:

| | Concentrations to test |
|---|---|
| | LysoTracker assay |
| HCR | 0.01-0.005-0.001% (of DE) |

b)—Evaluation of Lysosomal Network Protection
(i)—Observation of UV-irradiated control images allowed observing the effect of irradiation on cells shape and lysosomes organization in cells. Some UV-irradiated fibroblasts were indeed no more spindle-shaped and lysosomes network seemed less organized, compared to untreated and non-irradiated control.
(ii)—The measure of the fluorescence intensity corroborated this effect, as it significantly decreased: −29% (significant *). The application of a positive control (Vitamin C, 500 μM) diminished the effects of UVA. The results are shown in FIG. 3. Quantification data supported this observation, as fluorescence mean intensity was higher (highly significant effect **).
(iii)—The treatment of fibroblasts with the HCR extract at 0.01% protected lysosomes network damaged by UV irradiation. This effect against UV was less distinct with 0.005% and 0.001% of extract. This is confirmed by results of fluorescence quantification, as there was an increase of +36% with 0.01% HCR (significant *, compared to irradiated control). With lower tested doses (0.005% and 0.001%), effects were less distinct and not significant (+19% and +21% respectively).

c)—The present evaluation hence shows, that UVA irradiation causes partial damage of cell lysosomal network. Tested HCR extracts show effects on UV-irradiated fibroblasts and allows protecting the lysosomes network against UV irradiation, particularly at highest tested dose (0.01%). Accordingly, this evaluation permits to conclude that HCR extract is efficient to protect lysosomes of dermis cells against UV damage.

### EXAMPLE 4

### Evaluation of the Effects of HCR Extracts in a Human Skin Explants Pollution Model

### 1)—Background Information

a)—The aim of this study was to assess the potential protective effect of HCR extract, in a pollution model of human skin explants.

b)—The product was systemically applied for 24 hours, on human skin explants (diameter of 8 mm), provided by Biopredic™ International (Rennes), which derived from mammary plastic surgery (Caucasian origin forty-four years old woman);

c)—The product was then applied again during pollution, for another 24 hours. Said pollution was induced in this model by application of an environmental pollutant, benzo(a)pyrene (3,4-benzopyrene/BaP) as systemic treatment, i.e. in the culture medium. This pollutant induces IL-8 release (SEPhRA results).

d)—Release of the inflammatory cytokine IL-8 (interleukin 8) by the explants was analyzed by titration in the supernatants (culture media).

e)—In addition, skin explants were preserved for analysis by immunostainings of Claudins 1 & 4 expressions (CLDN1 and CLDN4).

### 2)—Human Skin Explants Model of Pollution

a)—A 2% (DE) HCR extract is diluted in the culture medium to obtain a 0.1% (DE) HCR extract, which is used as the test compound.

b)—Said HCR extract was systemically applied on the skin explants as a twenty-four hours pre-treatment.

c)—The skin explants were then polluted or not with BaP (Sigma CRM40071, 40 μM systemic) during another twenty-four hours, while the HCR extract was applied again systemically on both non-polluted and polluted skin explants. Each non-polluted condition was tested in triplicate (3 explants: 2 for immunostainings and 1 for histology), each polluted condition was tested in quadruplicate (4 explants: 3 for immunostainings and 1 for histology).

d)—Titrations in supernatants (removed for each condition, for the 3 non-polluted explants and the 4 polluted explants) were done in duplicate.

e)—In order to validate the IL-8 titration, an additional condition was evaluated, i.e. stimulation with a reference pro-inflammatory molecule PMA (Phorbol 12-myristate 13-acetate, Sigma P8139), at 1 μg/ml. This product was systemically applied during the last twenty-four hours on one explant not treated with the extract nor polluted.

### 3)—Treatments of the Skin Explants

a)—Skin explants were then placed in 24-wells plates filled with DMEM medium (Invitrogen™ 31053) added with 5% foetal calf/bovine serum (FBS, Invitrogen™ 10270) and with 300 μL/well antibiotics (penicillin 100 U/ml and streptomycin 100 μg/ml, Invitrogen™ 15140). Explants were stabilized during four hours at 37° C./5% CO₂.

b)—After stabilization, extract was systemically applied or not in the medium of the explants, that were incubated at 37° C./5% CO₂ during twenty-four hours. The medium used was then DMEM not added with FBS. Explants were then polluted or not with systemic application of BaP (40 μM) during twenty-four hours, while being treated again (or not) with the HCR extract (37° C./5% de CO₂ incubation).

c)—At the end of this period, supernatants (treatment media) were stored in order to dose IL-8. One explant of each condition (polluted or not) was preserved in 4% formaldehyde to perform the histological analysis (H/E/S staining). Other explants were cryo-preserved in liquid nitrogen, for later immunostaining experiments.

d)—The inflammatory cytokine IL-8 was then measured out with a commercial kit from Bio-Techne/R&D systems (D8000C), which is an ELISA method. In each case, the measure was done twice. Titration raw data were obtained by measuring the absorbance or the optical density (OD) at 450 nm wavelength, with 570 nm as a reference.

e)—The concentrations of IL-8 measured in the samples were determined by using a concentration range of an IL-8 standard. OD data obtained for IL-8 standards (pg/ml) were plotted in a standard curve with the concentrations on the abscissa axis and OD on the ordinate axis and using a calibration straight line IL-8 quantity (pg/ml or ng/ml) was determined from OD data by subtracting for each data the blank mean.

OD_{«sample»}=OD_{raw data}−OD mean_{«blank»}

Quantitative values of each condition were averaged and the IL-8 concentrations (ng/ml) are shown in FIG. 4.

### 4)—CLDN 1 and 4 Immunostainings

a)—The expression of these proteins was observed by fluorescent immunostaining, which a detection method involving the detection of the target protein with two antibodies: the first one, the primary antibody, is specific of the protein, so detects the protein; then, a secondary antibody, specific of the primary antibody, allows detecting the protein-primary antibody complex and emits fluorescence. In that manner, the expression profile of the target protein in the target tissue, here the epidermis skin, can be observed.

b)—Before immunostaining, the cryo-preserved explants were cut with a cryostat. Frozen cross-sections were mounted on slides and cryo-preserved.

c)—To start immunostaining, slides were defrosted and fixed with acetone. Non-specific fixation sites of the primary antibody were then blocked (“blocking”) with serum from the same species as the secondary antibody (Santa Cruz). After incubation at room temperature with the primary antibody that targets Claudin 1 or 4 (Santa Cruz sc-81796 or sc-17664 respectively), cross-sections were incubated with the secondary antibody coupled to a fluorochrome (Alexa type, Invitrogen™, 488 or 546 nm, green or red) and DAPI (Roche™), a blue fluorescent marker specific to DNA, to colour the nucleus.

d)—Finally, slides were “mounted”, with a mounting medium (Dako) that provides fluorescence protection and cover slips adhesion onto the slide. Slides were observed with ×40 objective under a fluorescence optic microscope (Olympus CK40) with appropriate filters to the fluorochromes (FITC green, or TRITC red) or to DAPI (blue). Images acquisition was done with Archimed logiciel (Microvision).

e)—For each of these two stainings, a specificity control of the primary antibody was done, the isotypic control. On one supplementary cross-section slide, instead of the primary antibody, a serum of non-specific immunoglobulins was used, from the same species of the primary antibody (IgG, Santa Cruz).

f)—A histology control was made to verify tissue integrity of skin explants, polluted or not, treated or not (check of the received tissues quality and absence of tissue structure deterioration by treatments). Explants were fixed and preserved in 4% formaldehyde at the end of treatment (one for each condition). Explants were then included in paraffin, cross-sections were mounted on slides, and coloured with hematoxyline/eosine/safran (or HES).

### 5)—Results

a)—Histological Analysis
(i)—A degradation of Claudin 1 (inhibition of expression and meshing failure) was observed in case of systemic application of the BaP pollutant.
(ii)—The HCR extract restored deteriorations caused by the pollutant.
(iii)—Application of BaP pollutant also degraded the expression of Claudin 4 (architecture damage, decrease of fluorescence intensity: thinner and less thick areas).
(iv)—Systemic application of the HCR extract restored the meshing failure caused by the pollutant.

b)—Titration of IL-8 Released from Skin Explants
(i)—IL-8 titration results after pollution or not, are shown in FIG. 4. Therein, data are expressed as IL-8 concentrations. Significance is indicated, compared to the unpolluted control (black stars), or compared to the polluted control (red stars).
(ii)—After pollution, the cytokine release was stimulated: +59% significant (*) (polluted control compared to the untreated/unpolluted control). With PMA stimulation (pro-inflammatory reference molecule), without pollution, the cytokine release was stimulated: +224% very highly significant (***) (compared to the untreated/unpolluted control).
(iii)—With the systemic application of HCR extract, the IL-8 release, was inhibited in both conditions: polluted or not. Without pollution, this effect was −69% and very highly significant (***, compared to the untreated/unpolluted control). With pollution, the IL-8 production, which was inducted by the pollutant, was inhibited by application of the HCR extract: −69%, very highly significantly (***, compared to the polluted control).

c)—The results demonstrate the efficiency of the HCR extract to fight against an inflammatory stress mediator that is inducted by pollution. Accordingly, it can concluded, that HCR extract is efficient to protect skin of an inflammatory stress inducted by a pollutant, and of degradations undergone by proteins that participate to maintain tissue integrity, homeostasis and epidermis barrier function.

### EXAMPLE 5

### Evaluation of the Effects of HCR Extracts on β-Endorphins Production by Normal Human Keratinocytes

### 1)—Background Information

The aim of this study was to evaluate in vitro effects of four HCR extracts on β-endorphins (or β-EP) by normal human keratinocytes (NHKs), extracted from abdominal skin epidermis of a 30 year-old donor (Caucasian woman). The β-endorphins are neuropeptides that were measured out in the culture supernatants by ELISA technique, after forty hours of treatment. A XTT assay was previously performed, to check the cells viability, in order to determine the concentrations to be tested, as described in Example 2.

### 2)—Dose Determination (Cytotoxicity/Viability Check by XTT Assay) and Evaluated Test Compound

a)—The dose determination with XTT assay was carried out as described in Example 2, but after a twenty-four hours treatment [instead of 48 hours in example § c)].

b)—Cells Seeding and Treatments

Cells were seeded at 12,500 keratinocytes/well (equivalent to 37,500 cells/cm², the cell density used later in the study), in 96-well microplates, in complete KSFM (Keratinocytes Serum Free Medium, with antibiotics and growth supplements), twenty-four hours before treatment (incubation at 37° C./5% CO₂). Each concentration of the test compound was tested in triplicate, in basal, i.e. non-supplemented medium (KSFM without growth supplements). Incubation time was twenty-four hours (at 37° C./5% CO₂).

### 3)—β-Endorphins Production by Keratinocytes

a)—NHKs were seeded in 24 wells plates and treated with different concentrations of the test compound, during forty hours. The β-EP were measured out in culture supernatants, by ELISA assay from USCN Life Science (CEA806Hu). The total proteins were also measured out, in cells pellets, in order to report β-EP titration to total proteins amount. dbAMPc (N-6, 2′-O-dibutyryladenosine 3′,3′-cyclicmonophosphate, Sigma D0627) at 2 mM was used as a positive control.

b)—Cell Treatment and β-EP Assay

Cells were seeded at 75 000 cells/well (37 500 cells/cm²), in 24 wells plates, in complete KSFM, 48 hours before treatment (incubation at 37° C./5% CO₂). Each concentration of the test compound was tested in triplicate, in basal KSFM, added with an anti-proteases cocktail of molecules (Leupeptine, Aprotinin and Phenylmethanesulfonyl fluoride or PMSF, Sigma L8511, A1153 and P7626 respectively). This cocktail permitted to protect the β-EP proteins until the titration. Incubation time was 40 hours (at 37° C./5% CO₂). Culture supernatants and cells pellets were collected and stored at −20° C. before testing.

c)—Absorbance data (or optical density, OD) were measured at 450 nm. Each sample was assayed in duplicate, so there were six values for each sample (except for untreated control and reference molecule, for which there were 8 and 2 values respectively).

d)—Proteins Titration (Bca Assay)

Total proteins titration in cell pellets was performed in parallel by colorimetric method based on bicinchoninic acid. The principle of the bicinchoninic acid (BCA) assay is similar to the Lowry procedure, in that both rely on the formation of a protein-Cu²⁺ complex under alkaline conditions, followed by reduction of the Cu²⁺ to Cu⁺. BCA is highly specific for Cu⁺ ion and forms a purple-blue complex with Cu⁺ in alkaline environments, thus providing a basis to monitor the reduction of alkaline Cu²⁺ by proteins. The amount of reduction and of BCA-Cu⁺ complex is proportional to the protein present and is quantified by spectrophotometric lecture (570 nm long wave). The proteins titration kit used (Sigma BCA1) is composed of a bicinchoninic acid solution (Sigma B9643) and a copper sulfate pentahydrate solution (CuSO₄—Sigma C2284). Standard range is prepared with BSA (Bovine Serum Albumin—Sigma A9418).

e)—Cells pellets were dry preserved at −20° C. awaiting cells lysis and titration. To lyse cells and alkalinize the reaction environment, cells pellets were equilibrated at room temperature and then lysed in alkaline solution during 30 minutes. Titration was realized adding a mix of the reagents (bicinchoninic acid+CuSO₄) to aliquots of lysates (cells pellets lysed). The plates were incubated at 37° C. and then lecture was performed at 570 nm longwave, after having stopped the reaction placing the plates at 4° C. for a few minutes.

f)—Expression of Results

For both β-EP and proteins titrations, OD data obtained for respective standards ere plotted on graphics to determine standard curves. Then, the amount/concentrations of proteins or β-EP measured in the samples can be determined. Quantitative values of each condition were averaged. The results are shown in FIGS. 5, 6 and 7 with amount/concentrations (pg/ml for β-EP and pg/well for total proteins).

### 4)—Results

a)—Dose Determination (Cytotoxicity/Viability Check by XTT Assay)

No cytotoxicity was observed for the tested concentrations between 0.0005% and 0.01% HCR (DE) on fibroblasts:

| | Concentrations to test (β-EP) |
|---|---|
| | (chosen according to XTT assay) |
| HCR extract | 0.01-0.005-0.001% |

b)—β-Endorphins Titration

Treatment with HCR extract generates a beneficial effect on β-EP release by keratinocytes, as an induction appears with two tested concentrations: +102%*** at 0.001% and +259%** at 0.005%. The results are shown in FIG. 5.

c)—Total Protein Titration

Reported to total proteins amount, the beneficial effect on β-EP release by keratinocytes observed after treatment with HCR extract is corroborated, since a stimulation is highlighted with two tested concentrations: +120%*** at 0.001% and +402%** at 0.005%.

Normal human keratinocytes, β-endorphins (β-EP) are produced and released in the culture supernatants in which the titration is done. Treatment of normal human keratinocytes with HCR extract (0.001 and 0.005%) induced stimulation of β-EP release.

### EXAMPLE 6

### Evaluation of the Effects of HCR Extracts on Melanogenesis in B16 Cells (Melanin Titration)

### 1)—Background Information

The aim of this study is to evaluate the effects of HCR extracts on melanogenesis in an in vitro model of murine B16 melanocytes. Cells used in this study were murine melanocytes (B16-F0 line, LGC standards, ATCC-CRL-6322) extracted from murine skin. This cells lines synthesizes a great quantity of melanin which is released in the culture media (so called supernatants), so a titration by spectrophotometry is easy to achieve. These melanocytes were cultured as monolayers; the test compounds were applied during seventy two hours and the synthesized melanin was then released and measured out in the supernatants. A XTT assay was previously performed, to check the cells viability, in order to determine the concentrations to be tested, as described in Example 2.

### 2)—Dose Determination (Cytotoxicity/Viability Check by XTT Assay) and Evaluated Test Compound

a)—The dose determination with XTT assay was carried out as described in Example 2, but after a seventy-two hours treatment [instead of 48 hours in example § c)].

b)—Cells Seeding and Treatments

Cells were seeded into 96-wells microplates (at a cell density of 10,500 cells per well, i.e. 31,250 B16/cm², the density used later in the study), 24 hours before treatment (incubation at 37° C./5% CO₂). Each concentration of the test compound was tested in sextuplicate. Incubation/treatment time was seventy-two hours (at 37° C./5% CO₂). Cells were seeded and treated in a Dulbecco's modified Eagle medium or DMEM, without phenol red (Gibco 11880), with antibiotics (Gibco 15140), L-glutamine (Gibco 25030) and supplemented with 10% of foetal bovine serum or FBS (Gibco 10270).

### 3)—Evaluation of Effect on Melanogenesis

a)—Assay Principle

B16 melanocytes were seeded in 24-wells plates. Then the cells were treated with the test compound (three concentrations), for seventy-two hours. Finally, the melanin and total proteins titrations were performed. Photos of cells culture every 24 hours also allowed the observation of melanin release all along the extract application (for 72 h). Positive controls were used in this study:
A reference molecule inhibiting melanogenesis (tyrosinase inhibitor), kojic acid (Sigma K3125) at 1 mM, and at the contrary,
A reference molecule that stimulates pigmentation (cAMP stimulator), an αMSH analog so called melanotan (Sigma M8764, [Nle4, D-Phe7]-α-Melanocyte Stimulating Hormone trifluoroacetate salt) at 100 nM.

The effects of these references were visible as soon as 48 h of treatment for both and also at 72 h for kojic acid. Three concentrations of the extract were tested (weight/volume % of dry extract or DE). These non-cytotoxic doses were tested according to the results obtained from the XTT assay.

b)—Cell Treatment and Melanin Titration

Cells were seeded into 24-wells plates (at a cell density of 60,000 cells per well, i.e. 31,250 B16/cm²), in the same medium as previously described, one day before treatment (incubation at 37° C./5% CO₂). Each concentration/dose of the test compound was then tested in triplicate for 72 h (incubation at 37° C./5% CO₂). Coloration of culture media was observed every 24 hours and compared to blanks (wells with treatment media but without cells). After 48 or 72 hours of treatment, the supernatants were homogenized and aliquots were transferred to 96-well plates for spectrophotometry reading at 492 nm wavelength (reading in duplicate). This titration permitted to quantify melanin liberated by the B16 cells, relatively to a standard range of synthetic melanin (M0418).

c)—Total Protein Titration (Bca Assay)

Total proteins titration in cell pellets was performed in parallel by colorimetric method based on bicinchoninic acid as described in Example 5.

d)—Expression of Results

For both melanin and proteins titrations, OD measurements obtained for respective standards were plotted on graphics to determine standard curves. Then, the amount/concentrations of proteins or melanin measured in the samples can be determined.

Quantitative values of each condition were averaged. The results with different concentrations of HCR extract are presented in FIGS. 8 to 13

### 4)—Results

a)—Dose Determination (Cytotoxicity/Viability Check by XTT Assay)

No cytotoxicity was observed for the tested concentrations between 0.0005% and

0.01% HCR (DE) on fibroblasts:

| | Concentrations to test (melanogenesis) | |
|---|---|---|
| HCR | 1^{st} experiment | 0.01-0.005-0.001% |
| (% of DE) | 2^{nd} experiment | 0.1-0.05-0.01% |

b)—Melanin Titration

In these experiments, the positive reference of depigmentation kojic acid (1 mM) induces a very strong and very highly significant (***) inhibition of the melanin release in the culture medium (more than 90%). After 48 h of treatment, the positive reference of pro-pigmentation melanotan (100 nM) induces a strong and highly significant (**) stimulation of the melanin release in the culture medium (more than two fold) (Results not shown in detail). At 72 h the melanine release is more distinct than at 48 h (OD more than 20-fold higher), which is normal and permits to observe potential pro-pigmentation effects at 48 h. The tested HCR extracts showed distinct depigmentation effects with the two highest tested concentrations after 72 h of treatment in this model. After 72 h of application in this model, HCR extract inhibited melanin release with a dose effect: −86% and −45% statistically very highly significant (***), respectively with 0.1% and 0.05% of DE (dry extract, which is equivalent to 1 mg/ml and 0.5 mg/ml). In the present evaluation, which represents a good model for melanogenesis by treatment of B16 melanocytes during 72 h, HCR extract permitted to inhibit melanin synthesis. Thus, the HCR extracts were found to be effective in reducing melanin liberation and consequently being able to prevent sun-induced pigmentary spots, i.e. irregular and non-uniform pigmentation.

### EXAMPLE 7

### Evaluation of the Antioxidant Potential of HCR Extracts in Normal Human Keratinocytes (ROS Detection with H₂DCFDA Probe)

### 1)—Background Information

The aim of the present evaluation was to evaluate the antioxidant and antipollution effects of the extract HCR, against oxidative stress in normal human cultured keratinocytes extracted from abdominal plastic surgery (62 year-old woman Caucasian donor), using the H₂DCFDA probe. The dichlorodihydrofluorescein diacetate probe (H₂DCFDA/dichlorofluorescin diacetate) is a chemically reduced form of fluorescein used as an indicator for reactive oxygen species (ROS) in cells. It is cell-permeant and stable.

When it penetrates the cell, acetate groups are cleaved by intracellular esterases. The resulting product is the dichlorodihydrofluorescein (or H₂DCF), non-fluorescent. In presence of ROS (O²⁻, OH, NO, ONOO, . . . ) i.e. with oxidation, it is converted to the highly fluorescent product, dichlorofluorescein (or DCF). The probe oxidation is not specific of one type of ROS. The fluorescence is detected at wavelengths of 490 nm (excitation) and 525 nm (emission). The oxidative stress is induced by an environmental pollutant, benzo(a)pyrene (3,4-benzopyrene/BaP) or by a reference generator of reactive oxygen species (ROS), hydrogen peroxide (oxygenated water/H₂O₂). A XTT assay was previously performed, to check the cells viability, in order to determine the concentrations to be tested, as described in Example 2.

### 2)—Evaluation of Antioxidant Effect (H₂DCFDA Probe)

a)—Assay Principle

Normal human keratinocytes were seeded in 96-well microplates and are then treated with the test compound to be tested for 24 hours, before adding the fluorescent probe for detection of ROS and stimulating or not by the pollutant or hydrogen peroxide. This test is based on the use of a detection system, a probe, which is degraded and fluoresces on contact with ROS. This probe H₂DCF-DA is cleaved of two CH₃—COOH groups by cellular esterases; then the probe meets ROS present in the cell and looses two hydrogen atoms to become fluorescent DCF.

b)—Cell Treatment and Melanin Titration

Cells were seeded in 96-well microplates at 10,000 keratinocytes/well (30,000 cells/cm²) in complete KSFM medium and were left to adhere for 24 hours at 37° C./5% CO₂. The cells were treated with 3 non-cytotoxic concentrations of the extract to be tested (in sextuplicate, in non-supplemented medium) for 24 hours and incubated at 37° C./5% CO₂, in parallel to control conditions (untreated control or antioxidant positive reference). An internal antioxidant positive reference was used in this study, used at 10 μM.

| | | Concentrations |
|---|---|---|
| | | to test |
| Without | Untreated control | / |
| stimulation | Positive control/ | 10 μM |
| | reference molecule | |
| | HCR extract | 0.01-0.005- |
| | | 0.001% of DE |
| | Acetone solvent control | 0.9% |
| With H₂O₂ | H₂O₂ Control | 1 mM |
| stimulation | Positive control + | 10 uM + 1 mM |
| (1 mM) | H₂O₂ | |
| With BaP | BaP control | 36 μM |
| stimulation | Positive control + BaP | 10 μM + 36 μM |
| (36 μM = | HCR extract + BaP | 0.01-0.005- |
| 9 μg/mL) | | 0.001% + 36 μM |

c)—Probe Incorporation

After washing cells, the probe (Invitrogen D399, diluted at 50 μM in non-supplemented KSFM) was applied and the plates were incubated for 45 minutes at 37° C./5% CO₂. Blanks wells were carried out without cells (but with the probe and tested products).

d)—ROS Stimulation

After washing the cells, BaP (Sigma CRM40071) or hydrogen peroxide solution (Sigma H1009) was applied in the expected wells (stimulated conditions, finally at 36 μM and 1 mM respectively, in non-supplemented KSFM). For other wells (non-stimulated conditions), medium alone was applied. Plates were incubated for 20 minutes at 37° C./5% CO₂.

e)—Expression of Results

After incubation, fluorescence was read at 490 nm (excitation) and 525 nm (emission) wavelengths (Tecan Safire II reader). Data are expressed as RFU (fluorescence units). Data are collected and the averaged blank data is subtracted.

RFU_{«sample»}=RFU_{raw data}−RFU mean_{«blank»}

### 3)—Results

Results of antioxidant potential assay with or without ROS stimulation (by hydrogen peroxide or BaP), are presented in FIGS. 14 and 15. Therein, data are expressed in fluorescence units (RFU). Significance (T-test) is indicated either comparatively to non-stimulated control (untreated of acetone solvent control: black stars), or comparatively to H₂O₂ or BaP-stimulated control (red stars). The blank controls (without cells but with test compounds) have permitted to verify absence of interaction between the tested extract or positive reference and H₂DCF-DA probe. Without ROS stimulation, there was no effect observed, in any of the different tests. With hydrogen peroxide treatment, cells produce free radicals, as there is a stimulation of +808% of ROS production (comparatively to unstimulated control). With BaP application, a stimulation is also observed, of +141% (comparatively to non-stimulated acetone solvent control). Concerning the reference molecule used, with ROS stimulation, an antioxidant effect is observed and is significant, as ROS production decreases by −21% (* with H₂O₂) and −33% (* with BaP). The tested HCR extract showed efficiency at a concentration of 0.01%, as this highest tested concentration permits to inhibit ROS production, stimulated by the BaP pollutant (diminution of ROS detected): −23%, nearly significant (p=0.122). The present evaluation hence showed that the tested HCR extract at the highest tested concentration (0.01%), shows antioxidant and antipollution potential.

### EXAMPLE 8

### Evaluation of the Effects of Hedychium coronarium Root Extract (HCR Extract) on Autophagic Activity in Fibroblasts Irradiated with Blue Light (MDC Assay)

### 1)—Background Information

The purpose of the study is to evaluate effects of one HCR extracts on autophagic activities of in fibroblast irradiated with 453 nanometers blue light. The cells (normal human fibroblasts or (NHF)) are cultured as primary monolayer. Product to be tested is applied during 24 hours before irradiation and the n the product to be tested is applied during 24 hours. then autophagic activity is measured (MDC).

Lysosomes are cytoplasmic organelles that permit recycling of cellular materials that have exceeded their lifetime or are otherwise no longer useful. Their main function is to digest endogeneous or exogeneous substrates (so called autophagy) in eukaryotic cells. Lysosomes break down cellular waste products, fats, carbohydrates, proteins, and other macromolecules into small compounds, which are transferred back into the cytoplasm as new cell-building materials. Indeed, its lipid membrane contains many enzymes, proton pumps and transport proteins. Acid pH is regulated to allow optimum activities of acid hydrolases [1]-[2]. The fluorescent LysoTracker probe is structured to easily insert in living cells (a fluorophore is linked to a weak base that is only partially protonated at neutral pH). This characteristic permits to this labeling/staining to be very selective for acid organelles i.e. the lysosomes [3-5]. Then, effects of radiations, like blue light for instance, at lysosomal level can be evaluated. Indeed, these radiations lead to free radical formation and affect the normal lysosomes network, its distribution in the cell space. This can be visualized with specific fluorescent probe LysoTracker. Autophagy is an eukaryotic cell process that allow cytoplasmic materials digestion in a vacuole that fuses with the lysosome. Indeed, lysosomes can directly incorporate cytoplasmic fragments (micro-autophagy), or via autophagic vacuoles, named autophagosomes (macro-autophagy, fusion of lysosome and autophagosome is so called autolysosome). Autophagy is an important mechanism that allows the cell to mobilize its energy stocks to defend and destroy its damaged organelles and then avoid serious effects. This process permits elimination and replacement of proteins and non-functional organelles, then to ensure homeostasis. It is an essential cytoprotective mechanism that allows adaptive cell responses to different kinds of stress or damages, as nutritive privation (starvation). So it is necessary to cell survival, but also to other fundamental physiologic phenomena as development, immunity, cell differentiation (including keratinocytes differentiation to corneocytes in the epidermis). It is involved in longevity control, aging, and development of pathologies as cancer or diabetes, infectious, metabolic, or neuro-degenerative diseases [6-7]. The MDC probe is specific of autophagic vacuoles and permits to easily and specifically quantify autophagic activity [8].

2)—Assay principles

a)—In order to assess the effects of the extract HCR on autophagic activity, fibroblasts are seeded in 96 wells plates and treated with the product, irradiated, treated again and then MDC probe permits to measure autophagic activity. Two positive references are used in this study, antioxidants known to protect from oxidative stress caused by UV irradiation: vitamin C (L-ascorbic acid, Sigma A4544) 500 μM (88 μg/mL) and vitamin E (α-tocopherol acetate, Sigma A1157) 200 μM (95 μg/mL). Three concentrations of the extract HCR are tested (weight/volume % of dry extract or DE, as it is a liquid extract), each dose being evaluated as quintuplicate (5 wells). Irradiation dose of 453 nm blue light is 40 J/cm² (23 mW/cm² for 29 minutes).

b)—Cells are seeded into 96-wells microplates (at a cell density of 10,000 cells per well, i.e.

30,000 NHF/cm²), 24 hours before treatment (incubation at 37° C./5% CO2). Cells are then treated with the extract for 24 hours (preventive effect), before being irradiated or not (453 nm BL, 40 J/cm², 23 mW/cm² for 29 minutes). Fibroblasts are then treated again with the product (curative effect) for another 24 h (so the total application is 48 h). Cells are seeded in complete medium: Dulbecco's modified Eagle medium or DMEM (Gibco 41966), with antibiotics (Gibco 15140) and 10% of foetal bovine serum or FBS (Gibco 10270). Treatments are done in medium without FBS. Irradiation is done in PBS (phosphate buffer saline). All incubation/treatment times are at 37° C./5% CO2 (except irradiation, done at room temperature).

c)—Autophagic Activity Assessment (MDC Assay)

At the end of treatment, wells are delicately rinsed (PBS) and 0.05 mM MDC probe is applied on cells (Sigma 30432) [17] (37° C./5% CO2). After incubation during 30 minutes and washes, fluorescence is measured at 380 nm (excitation) and 525 nm (emission) wavelengths (Tecan Safire II machine). Obtained data are expressed as RFU (real fluorescence units). A blank is done (wells containing probe but no cells).

### 3)—Results Expression

Blank mean is subtracted to all data and then for each condition RFU means are calculated. Significance is calculated comparing data to those obtained for suitable control (non-irradiated or irradiated), by Student t test (t-Test).

### 4)—Results

Results are indicated in Table 1 below:

TABLE 1
effect of HCR extract treatment of autophagy activity of fibroblast
irradiated cells by blue light (435 nanometers).

| | Quantity of | | RFU | |
|---|---|---|---|---|
| Product | treatment | | (means) | |
| Non treated and non irradiated NHF Cells | — | | 96 | RFU |
| HCR extract Treated and non irradiated NHF Cells | 0.001% | (w/w) | 94(*) | RFU |
| HRC extract Treated and non irradiated NHF Cells | 0.005% | (w/w) | 93(*) | RFU |
| HCR extract Treated and non irradiated NHF Cells | 0.01% | (w/w) | 96(*) | RFU |
| Vitamin C Treated and non irradiated NHF Cells | 500 | μMoles | 60 (***) | RFU |
| Vitamin E Treated and non irradiated NHF Cells | 200 | μMoles | 76 (**) | RFU |
| Non treated and irradiated (453 nanometers) | — | | 110 | RFU |
| NHF Cells | | | | |
| HCR Extract Treated and irradiated | 0.001% | | 96(*) | RFU |
| (453 nanometers) NHF Cells | | | | |
| HCR Extract Treated and irradiated | 0.005% | | 94(*) | RFU |
| (453 nanometers) NHF Cells | | | | |
| HCR Extract Treated and irradiated | 0.01% | | 71(***) | RFU |
| (453 nanometers) NHF Cells | | | | |
| Vitamin C Treated and irradiated | 500 | μMoles | 95 | RFU |
| (453 nanometers) NHF Cells | | | | |
| Vitamin E Treated and irradiated | 200 | μMoles | 86(*) | RFU |
| (453 nanometers) NHF Cells | | | | |

Student test:
(*)0.01 < p ≤ 0.05
(**): 0.001 < p ≤ 0.01
(***): p ≤ 0.001

### 5) Analysis and Comments

Results show that when fibroblast cells are irradiated by a blue light (wave length of 453 nanometers), the autophagic activity increases. When treated with vitamin C or vitamin E, and irradiated at 453 nanometers, autophagic activity of fibroblast cells decreases of 14% with vitamin C and of 22% with vitamin E. When treated with HCR extract at different weight concentrations, i.e. 0.001%, 0.005% and 0.01%, autophagic activity of said fibroblast cells decreases of respectively about 13%, 15% and 35%.

### BIBLIOGRAPHICAL REFERENCES

[1]: Gene expression profiling reveals aryl hydrocarbon receptor as a possible target for photobiomodulation when using blue light. Becker A, Klapczynski A, Kuch N, Arpino F, Simon-Keller K, De La Torre C, Sticht C, van Abeelen F A, Oversluizen G, Gretz N. Sci Rep. 2016; 6: 33847.
[2]: Animal cell structure. Lysosomes. Davidson M W. Molecular expressions [on line]. 2015. URL http://micro.magnet.fsu.edu/cells/lysosomes/lysosomes.html
[3]; Real-time visualization of photochemically induced fluorescence of 8-halogenated quinolones: lomefloxacin, clinafloxacin and Bay3118 in live human HaCaT keratinocytes. Koker E B, Bilski P J, Motten A G, Zhao B, Chignell C F, He Y Y. Photochem Photobiol. 2010; 86(4): 792-797.
[4]: An insight into the mechanisms of the phototoxic response induced by cyamemazine in cultured fibroblasts and keratinocytes. Morlière P, Haigle J, Aissani K, Filipe P, Silva J N, Santus R. Photochem Photobiol. 2004; 79(2): 163-71.
[5]: Screening of effective pharmacological treatments for MELAS syndrome using yeasts, fibroblasts and cybrid models of the disease. Garrido-Maraver J, Cordero M D, Moñino I D, et al. Br J Pharmacol. 2012; 167(6): 1311-1328.
[6]: Autophagy fights disease through cellular self-digestion. Mizushima N, Levine B, Cuervo A M, Klionsky D J. Nature. 2008; 451(7182):1069-75.
[7]: Autophagy and human diseases. Jiang P, Mizushima N. Cell Res. 2014; 24(1): 69-79.
[8]: Monodansylcadaverine (MDC) is a specific in vivo marker for autophagic vacuoles. Biederbick A, Kern H F, Elsasser H P. Eur J Cell Biol. 1995; 66(1): 3-14.

1. Composition comprising a plant extract of the specie Hedychium coronarium, for use in a method for treatment of the human body by therapy.

2. Composition comprising a Hedychium plant extract, for use in protecting and/or activating at least one of the cellular clean-up system in a method for treatment of the human body by therapy.

3. Composition according to claim 2, in which said clean-up system is selected from, the mitochondrial protection and/or activation, the lysosomal protection and/or activation, the inhibition of cellular IL-8 release, the cellular production of β-endorphins, the inhibition of irregular melanin release, and the protection and/or activation of the cellular transmembrane system by claudin restoration.

4. Composition according to claim 1 in which said plant extract of the specie Hedychium coronarium, is derived from flowers, seeds, fruits, leaves, stem, roots and/or rhizomes of the plant.

5. Composition according to claim 4, in which said plant extract of the specie Hedychium coronarium, is derived from roots and/or rhizomes of the plant.

6. A process for preparing a Hedychium coronarium roots and/or rhizomes extract according to claim 5, comprising the following steps:
A step a) which consists in providing a certain amount of pieces of roots and/or rhizomes of the Hedychium coronarium plant to be extracted;
A step b) during which said pieces of roots and/or rhizomes of the Hedychium coronarium plant are extracted with a suitable solvent or solvent mixture; to obtain an Hedychium coronarium plant roots and/or rhizomes pieces extraction mixture;
A step c) during which said Hedychium coronarium plant roots and/or rhizomes pieces extraction mixture obtained at step b), is filtrated to obtain a filtrate of Hedychium coronarium plant roots and/or rhizomes pieces extraction mixture;
A step d) during which said filtrate of Hedychium coronarium plant roots and/or rhizomes pieces extraction mixture obtained at step c), is let settle to separate the solid residues from the supernatant;
A step e) during which said supernatant obtained at step d) is filtrated to obtain a Hedychium coronarium plant roots and/or rhizomes extract as a clear solution;
A step f) during which said clear solution obtained at step e) is adjusted to the desired concentration using a suitable solvent to obtain the desired Hedychium coronarium roots and/or rhizomes extract.

7. The process according to claim 6, wherein said suitable solvent used in step f), is a mixture of water and glycerol.

8. The process according to claim 7, wherein the ratio water/glycerol (volume/volume) is equal to 30/70.

9. Composition according to claim 2 in which said plant extract of the specie Hedychium coronarium, is derived from flowers, seeds, fruits, leaves, stem, roots and/or rhizomes of the plant.

10. Composition according to claim 3 in which said plant extract of the specie Hedychium coronarium, is derived from flowers, seeds, fruits, leaves, stem, roots and/or rhizomes of the plant.
